(19) United States
(12) Patent Application Publication
(10) Pub. No.: US 2010/0285964 A1
(43) Pub. Date: Nov. 11, 2010

(54) SALTS OF 2-IODO-N-[(4-METHOXY-6-METHYL-1,3, 5-TRIAZINE-2-YL) CARBAMOYL] BENZENESULFONAMIDE, METHOD FOR THE PRODUCTION THEREOF AND USE THEREOF AS HERBICIDES AND PLANT GROWTH REGULATORS

(75) Inventors: Christian Waldraff, Bad Vilbel (DE); Klaus-Helmut Mueller, Duesseldorf (DE); Ernst Rudolf Gesing, Erkrath-Hochdahl (DE); Jan Dittgen, Frankfurt (DE); Dieter Feucht, Eschborn (DE); Hansjoerg Kraehmer, Hofheim (DE); Jeffrey Martin Hills, Idstein (DE); Georg Bonfig-Picard, Rodenbach (DE); Martin Hess, Mainz (DE); Dominique Schreiber, Frankfurt (DE); Hugh Christopher Rosinger, Hofheim (DE)

Correspondence Address: Baker Donelson Bearman, Caldwell & Berkowitz, PC, 920 Massachusetts Ave, NW, Suite 900, Washington, DC, 20001, US

(73) Assignee: BayerCropScience AG, Monheim (DE)

(21) Appl. No.: 12/738,366

(22) PCT Filed: Oct. 22, 2008
(86) PCT No.: PCT/EP2008/008947

§ 371 Date: Jul. 12, 2010

(30) Foreign Application Priority Data
Oct. 24, 2007 (EP) 07020807.9

(51) Int. Cl. A01N 43/66; C07D 251/42; C07C 265/00; C07C 263/16; A01P 13/00

(52) U.S. Cl. 504/227; 544/211; 560/330; 560/347

(57) ABSTRACT

The present invention relates to salts of 2-iodo-N-[(4-methoxy-6-methyl-1,3,5-triazin-2-yl)carbamoyl]benzenesulfonamide, to processes for their preparation and to their use as herbicides, in particular as herbicides for the selective control of unwanted harmful plants in crops of useful plants, permanent crops or on non-crop land, and also as plant growth regulators, on their own or with safeners and/or in combination with other herbicides, to their use for controlling unwanted harmful plants (such as, for example, broad-leaved/weed grasses) in specific crop plants or as crop protection regulators, for simultaneous and/or sequential application, either as a readymix or as a tank mix.

The present invention relates to salts of 2-iodo-N-[(4-methoxy-6-methyl-1,3,5-triazin-2-yl)carbamoyl]benzenesulfonamide, to processes for their preparation and to their use as herbicides, in particular as herbicides for the selective control of unwanted harmful plants in crops of useful plants, permanent crops or on non-crop land, and also as plant growth regulators, on their own or with safeners and/or in combination with other herbicides, to their use for controlling unwanted harmful plants (such as, for example, broad-leaved/weed grasses) in specific crop plants or as crop protection regulators, for simultaneous and/or sequential application, either as a readymix or as a tank mix.

It is known that substituted phenylsulfonylureas have herbicidal properties. These are, for example, phenyl derivatives which are mono- or polysubstituted (for example U.S. Pat. No. 4,127,405, WO 9209608, BE 853374, WO 9213845, EP 84020, WO 9406778, WO 02072560, U.S. Pat. No. 4,169,719, U.S. Pat. No. 4,629,494, DE 4038430). From WO 2006/114220, it is furthermore known that sulfonamides iodinated at the phenyl ring have herbicidal properties.

Surprisingly, it has now been found that salts of 2-iodo-N-[(4-methoxy-6-methyl-1,3,5-triazin-2-yl)carbamoyl]benzenesulfonamide are suitable in a particularly advantageous manner as herbicides and/or plant growth regulators.

Accordingly, the present invention provides agrochemically active salts of 2-iodo-N-[(4-methoxy-6-methyl-1,3,5-triazin-2-yl)carbamoyl]benzenesulfonamide.

The present invention preferably provides compounds of the general formula (I)

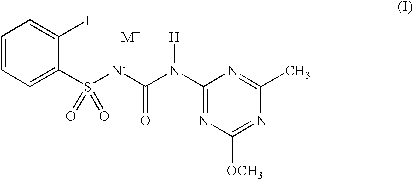

where the cation (M⁺)
(a) is an alkali metal ion, preferably lithium, sodium, potassium or
(b) is an alkaline earth metal ion, preferably calcium or magnesium, or
(c) is a transition metal ion, preferably manganese, copper, zinc or iron, or
(d) is an ammonium ion in which optionally one, two, three or all four hydrogen atoms are substituted by identical or different radicals from the group consisting of (C₁-C₄)-alkyl, hydroxy-(C₁-C₄)-alkyl, (C₃-C₆)-cycloalkyl, (C₁-C₄)-alkoxy-(C₁-C₄)-alkyl, hydroxy-(C₁-C₄)-alkoxy-(C₁-C₄)-alkyl, (C₁-C₆)-mercaptoalkyl, phenyl and benzyl, where the radicals mentioned above are optionally substituted by one or more identical or different radicals from the group consisting of halogen, such as F, Cl, Br or I, nitro, cyano, azido, (C₁-C₆)-alkyl, (C₁-C₆)-haloalkyl, (C₃-C₆)-cycloalkyl, (C₁-C₆)-alkoxy, (C₁-C₆)-haloalkoxy and phenyl, and where in each case two substituents at the nitrogen atom together optionally form an unsubstituted or substituted ring, or
(e) is a phosphonium ion, or
(f) is a sulfonium ion, preferably tri-((C₁-C₄)-alkyl)sulfonium, or
(g) is an oxonium ion, preferably tri-((C₁-C₄)-alkyl)oxonium, or
(h) is a saturated or unsaturated/aromatic nitrogenous heterocyclic ionic compound which has 1-10 carbon atoms in the ring system and is optionally mono- or polycondensed and/or mono- or polysubstituted by (C₁-C₄)-alkyl.

Preference is furthermore given to compounds of the formula (I) in which the cation (M⁺)
(a) is an alkali metal ion, preferably lithium, sodium, potassium or
(b) is an alkaline earth metal ion, preferably calcium or magnesium, or
(c) is a transition metal ion, preferably manganese, copper, zinc or iron, or
(d) is an ammonium ion in which optionally one, two, three or all four hydrogen atoms are substituted by identical or different radicals from the group consisting of (C₁-C₄)-alkyl, hydroxy-(C₁-C₄)-alkyl, (C₃-C₄)-cycloalkyl, (C₁-C₂)-alkoxy-(C₁-C₂)-alkyl, hydroxy-(C₁-C₂)-alkoxy-(C₁-C₂)-alkyl, (C₁-C₂)-mercaptoalkyl, phenyl and benzyl, where the radicals mentioned above are optionally substituted by one or more identical or different radicals from the group consisting of halogen, such as F, Cl, Br or I, nitro, cyano, azido, (C₁-C₂)-alkyl, (C₁-C₂)-haloalkyl, (C₃-C₄)-cycloalkyl, (C₁-C₂)-alkoxy, (C₁-C₂)-haloalkoxy and phenyl, and where in each case two substituents at the nitrogen atom together optionally form an unsubstituted or substituted ring, or
(e) is a quaternary phosphonium ion, preferably tetra-((C₁-C₄)-alkyl)phosphonium or tetraphenylphosphonium, where the (C₁-C₄)-alkyl radicals and the phenyl radicals are optionally mono- or polysubstituted by identical or different radicals from the group consisting of halogen, such as F, Cl, Br or I, (C₁-C₂)-alkyl, (C₁-C₂)-haloalkyl, (C₃-C₄)-cycloalkyl, (C₁-C₂)-alkoxy and (C₁-C₂)-haloalkoxy, or
(f) is a tertiary sulfonium ion, preferably tri-((C₁-C₄)-alkyl)sulfonium or triphenylsulfonium, where the (C₁-C₄)-alkyl radicals and the phenyl radicals are optionally mono- or polysubstituted by identical or different radicals from the group consisting of halogen, such as F, Cl, Br or I, (C₁-C₂)-alkyl, (C₁-C₂)-haloalkyl, (C₃-C₄)-cycloalkyl, (C₁-C₂)-alkoxy and (C₁-C₂)-haloalkoxy, or
(g) is a tertiary oxonium ion, preferably tri-((C₁-C₄)-alkyl)oxonium, where the (C₁-C₄)-alkyl radicals are optionally mono- or polysubstituted by identical or different radicals from the group consisting of halogen, such as F, Cl, Br or I, (C₁-C₂)-alkyl, (C₁-C₂)-haloalkyl, (C₃-C₄)-cycloalkyl, (C₁-C₂)-alkoxy and (C₁-C₂)-haloalkoxy, or
(h) is a cation from the group of the following heterocyclic compounds, such as, for example, pyridine, quinoline, 2-methylpyridine, 3-methylpyridine, 4-methylpyridine, 2,4-dimethylpyridine, 2,5-dimethylpyridine, 2,6-dimethylpyridine, 5-ethyl-2-methylpyridine, piperidine, pyrrolidine, morpholine, thiomorpholine, pyrrole, imidazole, 1,5-diazabicyclo[4.3.0]non-5-ene (DBN), 1,8-diazabicyclo[5.4.0]undec-7-ene (DBU).

Preference is given to compounds of the formula (I) in which the cation (M⁺) is a sodium ion, a potassium ion, a lithium ion, a magnesium ion, a calcium ion, an NH₄⁺ ion, a (2-hydroxyeth-1-yl)ammonium ion, a bis-N,N-(2-hydroxyeth-1-yl)ammonium ion, a tris-N,N,N-(2-hydroxyeth-1-yl)ammonium ion, a methylammonium ion, a dimethylammonium ion, a trimethylammonium ion, a tetramethylammonium ion, an ethylammonium ion, a diethylammonium ion, a triethylammonium ion, a tetraethylammonium ion, an isopropylammonium ion, a diisopropylammonium ion, a tetrapropylammonium ion, a tetrabutylammonium ion, a 2-(2-hydroxyeth-1-oxy)eth-1-ylammonium ion, a di(2-hydroxyeth-1-yl)ammonium ion, a trimethylbenzylammonium ion, a tri-((C₁-C₄)-alkyl)sulfonium ion or a tri-((C₁-C₄)-alkyl)oxonium ion, a benzylammonium ion, a 1-phenylethylammonium ion, a 2-phenylethylammonium ion, a diisopropylethylammonium ion, a pyridinium ion, a piperidinium ion, an imidazolium ion, a morpholinium ion, a 1,8-diazabicyclo[5.4.0]undec-7-enium ion.

Preference is furthermore given to compounds of the formula (I) in which the cation (M⁺) is a sodium ion, a potassium ion, a magnesium ion, a calcium ion or an NH₄⁺ ion.

Particular preference is given to compounds of the formula (I) in which the cation (M⁺) is a sodium ion, a potassium ion or an NH₄⁺ ion.

Very particular preference is given to compounds of the formula (I) in which the cation (M⁺) is a sodium ion or a potassium ion.

In the formula (I) and in all subsequent formulae, the carbon-containing radicals, such as alkyl, alkoxy, may in each case be straight-chain or branched, for example methyl, ethyl, n- or i-propyl, n-, i-, t- or 2-butyl.

Cycloalkyl is a carbocyclic saturated ring system having preferably 3-6 carbon atoms, for example cyclopropyl, cyclobutyl, cyclopentyl or cyclohexyl.

Depending on the nature and the attachment of the substituents, the compounds of the formula (I) may be present as stereoisomers. The formula (I) embraces all possible stereoisomers defined by their specific spatial form, such as enantiomers, diastereomers, Z and E isomers.

If, for example, one or more alkenyl groups are present, diastereomers (Z and E isomers) may occur. If, for example, one or more asymmetric carbon atoms are present, enantiomers and diastereomers may occur. Stereoisomers may be obtained from the mixtures resulting from the preparation using customary separation methods, for example by chromatographic separation techniques. It is also possible to prepare stereoisomers selectively by using stereoselective reactions employing optically active starting materials and/or auxiliaries. Accordingly, the invention also relates to all stereoisomers embraced by the general formula (I) but not shown in their specific stereoform, and to their mixtures.

The above examples of radicals or ranges of radicals which are subsumed under the general terms such as “alkyl” do not constitute a complete enumeration. The general terms also embrace the definitions, given later on below, of radical ranges in groups of preferred compounds, especially radical ranges which embrace specific radicals from the tabular examples.

The general or preferred radical definitions given above apply both to the end products of the formula (I) and, correspondingly, to the starting materials or intermediates required in each case for the preparation. These radical definitions can be combined with one another as desired, i.e. including combinations between the given preferred ranges.

The present invention also provides processes for preparing the salts according to the invention, in particular of compounds of the general formula (I), wherein
a) 2-iodobenzenesulfonamide (II)

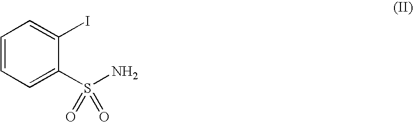

is reacted with a heterocyclic carbamate of the formula (III)

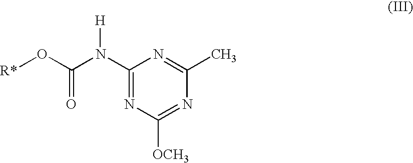

in which R* is a substituted or unsubstituted (C₁-C₂₀)-hydrocarbon radical, such as aryl or alkyl, preferably optionally substituted phenyl or optionally substituted (C₁-C₄)-alkyl,
or
b) a sulfonylcarbamate of the formula (IV)

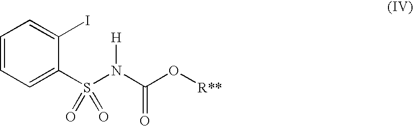

in which R** is a substituted or unsubstituted (C₁-C₂₀)-hydrocarbon radical, such as aryl or alkyl, preferably optionally substituted phenyl or optionally substituted (C₁-C₄)-alkyl,
is reacted with a 2-amino-4-methoxy-6-methyltriazine of the formula (V)

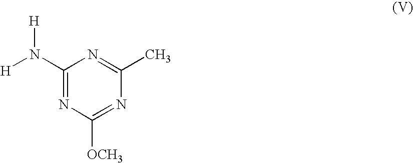

or
c) 2-iodobenzenesulfonyl isocyanate (VI)

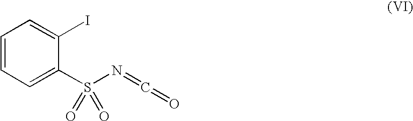

is reacted with the aminoheterocycle of the formula (V), or
d) 2-iodobenzenesulfonamide (II) is reacted in the presence of a base with the isocyanate (VII)

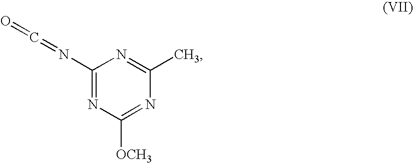

or
e) the aminoheterocycle of the formula (V) is initially reacted under base catalysis with a carbonic ester, for example diphenyl carbonate, and the intermediate formed is reacted in a one-pot reaction with 2-iodobenzenesulfonamide (II) (see variante a)) (cf. JP1989221366), or
f) a 2-iodobenzenesulfonyl halide of the formula (VIII)

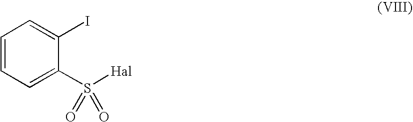

where Hal is a halogen atom, preferably chlorine (VIIIa) or fluorine (VIIIb) or bromine (VIIIc), is reacted with a cyanate, for example a metal cyanate, in particular an alkali metal cyanate, such as sodium cyanate, to give the isocyanate of the formula (VI) or a solvated (stabilized) derivative thereof, and subsequently reacted with the aminoheterocycle of the formula (V),
g) 2-iodobenzenesulfonamide (II) is reacted with a heterocyclic biscarbamate of the formula (IIIa)

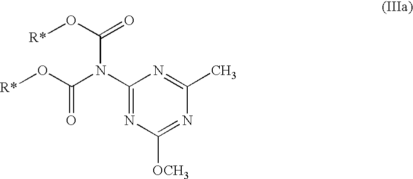

in which R* is a substituted or unsubstituted (C₁-C₂₀)-hydrocarbon radical, such as aryl or alkyl, preferably optionally substituted phenyl or optionally substituted (C₁-C₄)-alkyl (see WO 96/22284),
h) 2-iodobenzenesulfonamide (II) is initially reacted under base catalysis with a carbonic ester, for example diphenylcarbamate, and the intermediate formed is reacted in a one-pot reaction with the aminoheterocycle of the formula (V) (see variant b)).

The reaction of the compounds of the formulae (II) and (III) according to variant a) is preferably carried out under base catalysis in an inert organic solvent, such as, for example, dichloromethane, acetonitrile, dioxane or THF, at temperatures between 0° C. and the boiling point of the solvent, preferably at room temperature. The bases used here are, for example, organic amine bases, such as 1,8-diazabicyclo[5.4.0]-undec-7-ene (DBU), alkali metal tert-butoxides, such as, for example, NaO-tert-butoxide, or alkali metal hydroxides, such as, for example, NaOH, in particular for R*=(subst.) phenyl (cf. EP-A-44 807), or trialkylaluminum, such as trimethylaluminum or triethylaluminum, the latter in particular for R*=alkyl (cf. EP-A-166 516). Here, the base in question is employed, for example, in a range of from 1 to 3 molar equivalents, based on the compound of the formula (II).

2-Iodobenzenesulfonyl isocyanate is a novel compound which, like its preparation and its use for preparing compounds of the formula (I), forms part of the subject matter of the present invention.

2-Iodobenzenesulfonamide (II) can be obtained, for example, as shown in schemes 1 to 7 below.

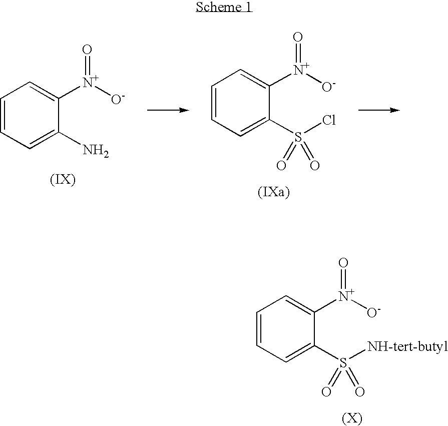

Starting with commercially available 2-nitroaniline (IX), it is possible to obtain, for example by diazotization of the amino group with an alkali metal nitrite, such as, for example, sodium nitrite, in the presence of hydrochloric acid at temperatures between −10° C. and 10° C. and subsequent exchange of the resulting diazo group, for example with sulfur dioxide in the presence of a diluent, such as, for example, dichloromethane, 1,2-dichloroethane or acetic acid, and in the presence of a catalyst, such as, for example, copper(I) chloride and/or copper(II) chloride, at temperatures between −10° C. and 50° C., 2-nitrobenzenesulfonyl chloride (IXa) (cf. Meerwein, Chem. Ber. 1957, 90, 841) (Scheme 1). Alternatively to the use of sulfur dioxide, it is also possible to employ Na₂S₂O₅ (sodium metabisulfite) as SO₂ source. By treating (IXa) with tert-butylamine, it is possible to obtain N-tert-butyl-2-nitrobenzenesulfonamide (X). The sulfonamide formation is carried out, for example, in inert solvents, such as, for example, dichloromethane, tetrahydrofuran (THF), dioxane, toluene or dimethylformamide (DMF), at temperatures between −70° C. and the boiling point of the solvent used, preferably at 25° C. Here, it is preferred to use the amine in an amount of 1.5-2.5 equivalents, based on the sulfonyl chloride employed.

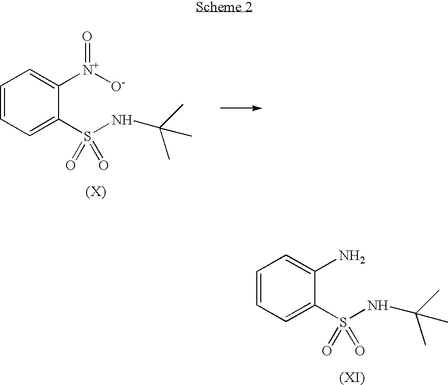

The reduction of (X) to N-tert-butyl-2-aminobenzenesulfonamide (XI) is carried out analogously to known methods (cf. Houben-Weyl, “Methoden der Organischen Chemie” [Methods of Organic Chemistry], 4th edition volume XI/1 pp. 360 ff., Thieme Verlag Stuttgart, 1957) (Scheme 2).

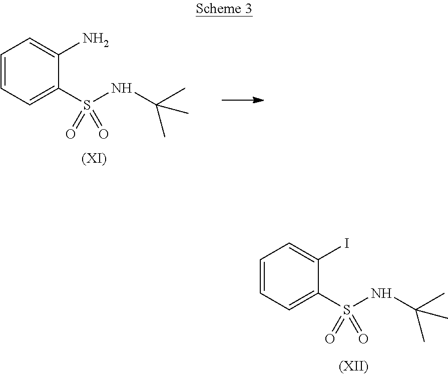

Aniline (XI) can be diazotized under conditions customary for diazotization reactions and then be converted into N-tert-butyl-2-iodobenzenesulfonamide (XII). The diazotization is carried out, for example, in the presence of the acid H⁺X⁻, where X⁻ is preferably Cl⁻, I⁻ or HSO₄⁻, in aqueous solution, if appropriate using an organic solvent inert under the reaction conditions, with a nitrite. The diazotization is carried out, for example, using an alkali metal nitrite such as NaNO₂ (sodium nitrite) in amounts of 1.0-1.2 mol of nitrite, preferably 1.01-1.05 mol of nitrite, per mole of aniline (XI). Suitable acids are mineral acids or strong organic acids; preference is given to hydrochloric acid or sulfuric acid. The solvent is water or a mixture of water and an organic solvent inert under the reaction conditions. The reaction temperature is generally between −5° C. and 50° C., preferably from 10° C. to 20° C.

The conversion of the diazonium salts obtained into the iodine compound (XII) is generally carried out without isolation and in the same aqueous or aqueous/organic solvent or solvent mixture as the diazotization. During the reaction, the diazonium group is exchanged for the iodine atom, either by the anion of the diazonium salt (if in the acid X⁻=I⁻) or (if X⁻ is not I⁻) by the reaction with added iodide, for example alkali metal iodide, preferably sodium iodide or potassium iodide. Here, the amount of iodide is, for example, from 1.1 to 1.5 mol of iodide per mole of aniline (XI) originally employed. Here, the reaction temperature is generally from 10° C. to 40° C., preferably from 15° C. to 30° C. (cf., for example, DE 19625831 and Bioorg. Med. Chem. 2004, 12, 2079) (Scheme 3).

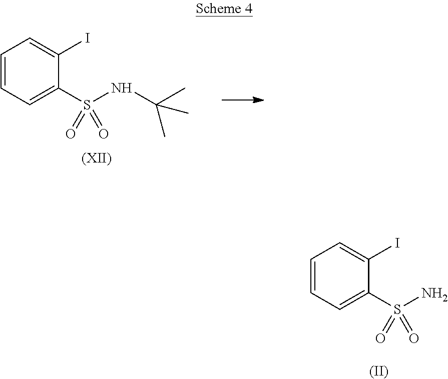

The removal of the tert-butyl protective group in (XII) to give 2-iodobenzene-sulfonamide (II) is carried out, for example, by treatment with a strong acid (see WO 89/10921). Suitable strong acids are, for example, mineral acids, such as H₂SO₄ or HCl, or strong organic acids, such as trifluoroacetic acid. The reaction is carried out, for example, at temperatures of from −20° C. to the respective reflux temperature of the reaction mixture, preferably at from 0° C. to 40° C. The reaction can be carried out neat or else in an inert solvent, such as, for example, dichloromethane or trichloromethane (Scheme 4).

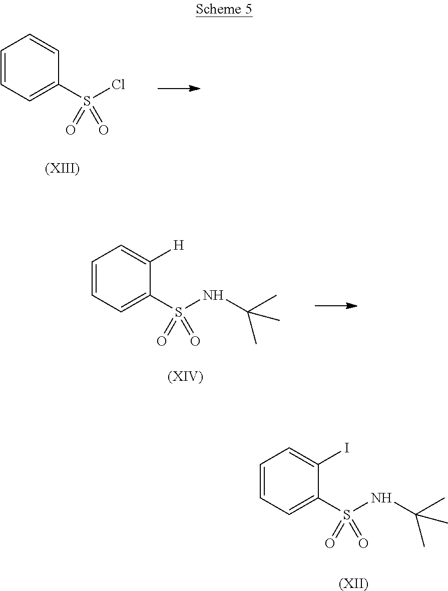

N-tert-Butyl-2-iodobenzenesulfonamide (XII) can also be obtained by metallating N-tert.-butylbenzenesulfonamide (XIV), which can be obtained by reacting commercially available benzenesulfonyl chloride (XIII) with tert-butylamine (see Scheme 1), with an organometallic compound, such as, for example, alkyl- or aryllithium, preferably n- or sec-butyllithium in hexane, if appropriate in the presence of a (further) inert diluent, such as, for example, tetrahydrofuran, and under an atmosphere of inert gas, such as, for example, under argon or nitrogen, at temperatures between −70° C. and 20° C.—i.e. the hydrogen atom located in the ortho-position to the SO₂NH-tert-butyl group in (XIV) is exchanged for a metal atom—and then reacting in the same reaction medium with iodine at temperatures between −100° C. and 40° C., preferably between −70° C. and 20° C., whereby the metal atom is replaced by iodine (Scheme 5) (see also: V. Snieckus et al., J. Org. Chem. 2001, 66, 3662 and Synlett 2000, (9), 1294).

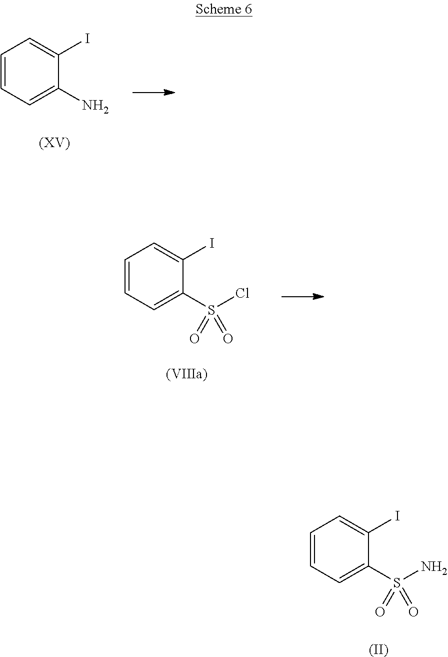

2-Iodobenzenesulfonamide (II) can also be obtained (Scheme 6) by reacting 2-iodobenzenesulfonyl chloride (VIIIa), which is prepared by diazotization of the amino group in 2-iodoaniline (XV) and subsequent exchange of the resulting diazo group for a chlorosulfonyl group (as described in more detail in scheme 1), with ammonia. To this end, ammonia gas is introduced into inert solvents, preferably dichloromethane or tetrahydrofuran, until no more ammonia is taken up. Alternatively, 2-iodobenzensulfonyl chloride (VIIIa) can also be converted into the N-tert-butyl-2-iodobenzenesulfonamide (XII) by reaction with tert-butylamine (analogously to Schemes 1 to 5). The tert-butyl protective group is then removed using acid (analogously to Scheme 4), to give 2-iodobenzenesulfonamide (II).

The sulfonylcarbamates of the general formula (IV) are prepared analogously to reactions known per se (cf. EP-A-120 814). It is also possible, for example, to convert 2-iodobenzenesulfonyl isocyanate of the formula (VI) in a smooth reaction in an inert solvent, preferably diethyl ether or dichloromethane, with phenol into the carbamates of the formula (IV). The aminoheterocycles of the formula (V) are known chemicals for synthesis, some of which are commercially available.

The reaction of the sulfonylcarbamates of the formula (IV) with the aminoheterocycles of the formula (V) is carried out by known processes (cf., for example, WO 2003 091228) (Scheme 7).

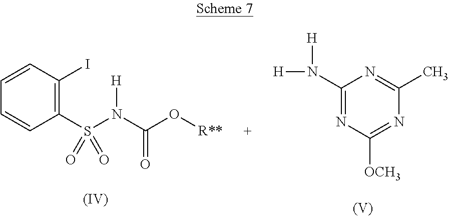

2-Iodobenzenesulfonyl isocyanate of the formula (VI) is a novel compound and accordingly also forms part of the subject matter of the present invention. It can be prepared by processes known per se from 2-iodobenzenesulfonamide (II) (cf. DE 3208189, EP 23422, EP 64322, EP 44807, EP 216504). The sulfonyl isocyanate of the formula (VI) is obtained when 2-iodobenzenesulfonamide (II) is reacted with phosgene, diphosgene or triophosgene, if appropriate in the presence of an alkyl isocyanate, such as, for example, butyl isocyanate, if appropriate in the presence of a reaction auxiliary, such as a tertiary amine, preferably a diazobicyclo[2.2.2]octane, and in the presence of a diluent, such as toluene, xylene or chlorobenzene, at temperatures between 80° C. and 150° C., and the volatile components are, if appropriate, distilled off under reduced pressure once the reaction has ended (Scheme 8).

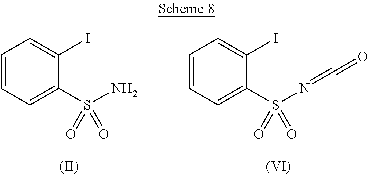

The reaction of 2-iodobenzenesulfonyl isocyanates of the formula (VI) with the aminotriazine of the formula (V) is carried out, for example, according to known processes (cf. WO 2003 091228) (Scheme 9).

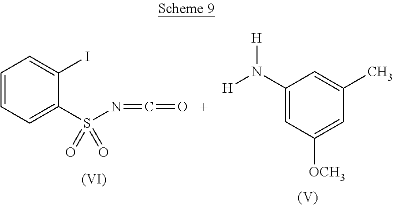

The isocyanates of the general formula (VII) are obtained, for example, from the aminoheterocycles of type (V) by treatment with oxalyl chloride or phosgene (analogously to Angew. Chem. 1971, 83, p. 407; EP 388 873). The reaction of the isocyanate type (VII) with 2-iodobenzenesulfonamide (II) is carried out, for example, analogously to variant c) (Scheme 10).

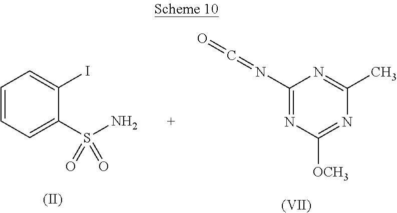

2-Iodobenzenesulfonyl fluoride (VIIIb) can be prepared by various methods known from the literature: i) from 2-iodobenzenesulfonamide (II) by diazotization with alkali metal nitrite, for example sodium nitrite, and subsequent reaction with hydrogen fluoride (J. Am. Chem. Soc. 1951, 73, 1857); ii) by reacting 2-iodobenzenesulfonyl chloride (VIIIa) with potassium fluoride (J. Chem. Soc, Perkin Trans. 1, 1998, 5, 875); iii) by reacting 2-iodobenzenesulfonic acid (XVII) with fluorosulfonic acid (U.S. Pat. No. 2,686,202).

2-Iodobenzenesulfonyl bromide (VIIIc) can be synthesized, for example, by reacting 2-iodobenzenesulfonyl chloride (VIIIa) with hydrogen bromide in acetic acid (Dokl. Akad. Nauk SSR 1955, 103, 627).

In one embodiment of variant f), the reaction mixture obtained by reacting the sulfonyl halide (VIII) with a cyanide is used directly for the coupling with the aminotriazine of the formula (V) for synthesizing of the precursor (neutral compound) of the formula (I) (cf. WO 2003 091228 and U.S. Pat. No. 5,550,238).

The salts according to the invention, in particular those of the formula (I), can be prepared from the neutral form of the sulfonylurea or sulfonylurea metal salts, in particular alkali metal salts (see, for example, EP-A-30138, EP-A-7687), or else from sulfonamide salts, for example in the manner below:

1. Deprotonation of the neutral sulfonylurea (XX) with a suitable base of the formula M⁺B⁻ (Scheme 11), where B⁻ is, for example, hydride, hydroxy or alkoxy anions, such as methoxy, ethoxy, n-propoxy, isopropoxy, n-butoxy or t-butoxy.

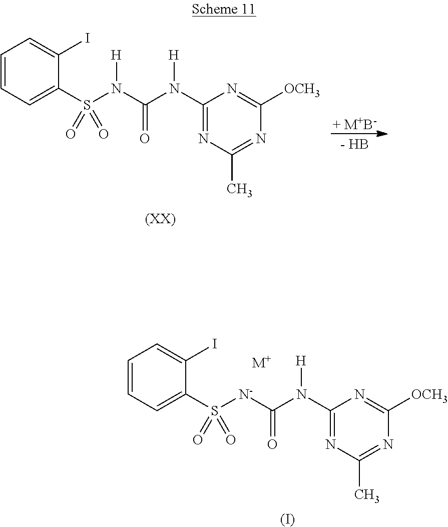

To this end, the sulfonylurea of the formula (I) is dissolved or suspended in an inert solvent or solvent mixture and reacted with one equivalent of M⁺B⁻ at temperatures between −20° C. and 100° C., preferably between −10° C. and 50° C.

2. Reaction of metal salts of the sulfonylurea of the formula (Ia) in which Met⁺ is a metal cation, preferably an alkali metal ion, such as Na⁺ or K⁺, with suitable reagents of the formula M⁺X⁻ (Scheme 12), where M⁺ is an ammonium ion and X⁻ may be an anion, for example a halogen anion, such as F, Cl⁻ or Br⁻, or a phosphate, sulfate or carboxylate anion, where this definition includes inorganic and organic salts as customary, for example, in surfactant chemistry (for example organic phosphate anions, phosphonate anions, sulfate anions, sulfonate anions, carboxylates).

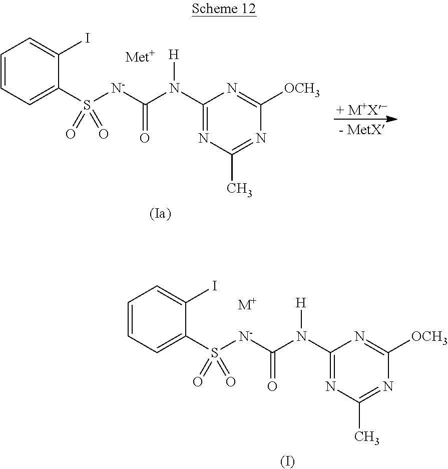

To this end, the metal salts according to the invention, for example alkali metal salts (for example Met⁺=Na⁺, K⁺) of the sulfonylurea are dissolved in an inert solvent or solvent mixture and reacted with one equivalent of the reagent M⁺X⁻. After the reaction has ended, the salt, for example alkali metal salt (such as NaCl), which is obtained as a by-product can be removed by filtration.

3. In situ deprotonation and cation exchange (Scheme 13) starting with neutral sulfonylurea (XX) using suitable reagents a) of the formula M⁺X⁻, where M⁺ is an ammonium ion and X⁻ may be an anion, for example a halogen anion, such as F, Cl⁻ or Br⁻, or a phosphate, sulfate or carboxylate anion, this definition including inorganic and organic salts, as customary, for example, in surfactant chemistry (for example organic phosphate anions, phosphonate anions, sulfate anions, sulfonate anions, carboxylates) and b) of the formula Met⁺B⁻, where Met⁺ is a metal cation, in particular an alkali metal cation, such as Na⁺ or K⁺, and B⁻ is a suitable base, for example a hydroxy or alkoxy anion, such as methoxy, ethoxy, n-propoxy, isopropoxy or n-butoxy or the anion of an alkoxylated, for example ethoxylated or propoxylated, (C₈-C₄₀)-alcohol.

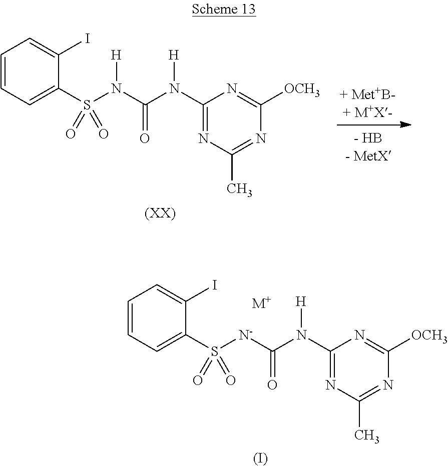

To this end, the neutral sulfonylurea (XX) is dissolved in an inert solvent or solvent mixture and reacted with one equivalent each of the reagents M⁺X⁻ and MetB. After the reaction has ended, the metal salt, in particular alkali metal salt (for example NaCl), obtained as a by-product can be removed by filtration.

4. Reaction of the neutral sulfonylurea (XX) with suitable zwitterions, for example (Scheme 14)

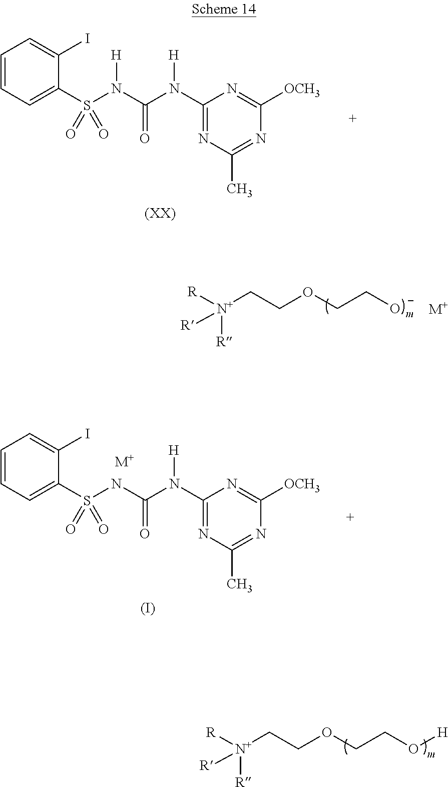

where R is a (C₁-C₂₀)-carbon radical, such as (C₁-C₁₀)-alkyl, R′ and R″ are identical or different and are hydrogen or (C₁-C₃₀)-hydrocarbon radicals, such as (C₁-C₁₀)-alkyl, and m is an integer from 0 to 100.

To this end, the neutral sulfonylurea (XX) is reacted with a zwitterion, as shown, for example, in Scheme 14, in an inert solvent, for example methanol, tetrahydrofuran or methylene chloride, or in a solvent mixture, at temperatures between −20° C. and 100° C., preferably between −10° C. and 80° C., in equimolar ratios.

5. Reaction of a sulfonamide salt of the formula (IIa) with the isocyanate (VII) (Scheme 15).

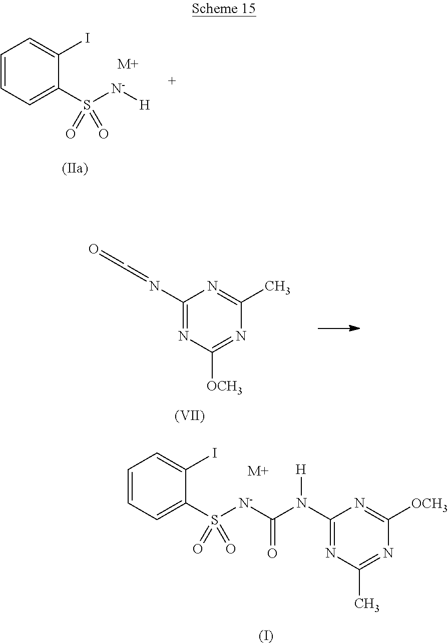

The reaction is carried out in an inert solvent or solvent mixture—such as, for example, tetrahydrofuran—at temperatures between −20° C. and 100° C., preferably between −10° C. and 70° C., by reacting the isocyanate (VII) in equimolar amounts with the sulfonamide salt of the formula (IIa). Here, the sulfonamide salt of the formula (IIa) can be employed directly or formed in situ—for example by reacting the corresponding sulfonamide of the formula (II) with a suitable base M⁺X⁻, where M⁺ is an ammonium ion and X⁻ is, for example, a hydroxy or alkoxy anion.

6. Reaction of a sulfonamide salt of the formula (IIa) with a carbamate of the general formula (III) (Scheme 16)

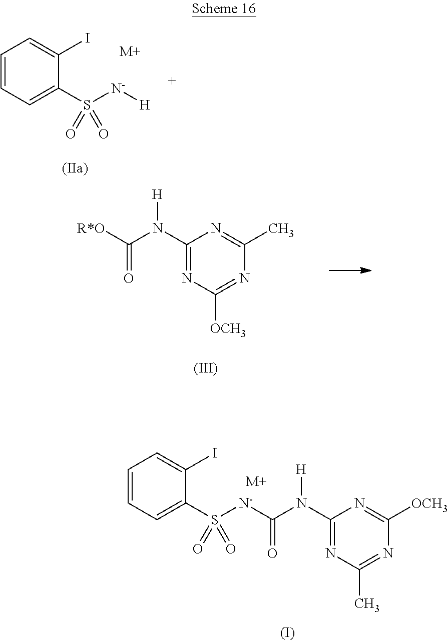

The reaction is carried out in an inert solvent (or solvent mixture)—such as, for example, tetrahydrofuran—at temperatures between −20° C. and 100° C., preferably between −10° C. and 70° C., by reacting the carbamate of the formula (III) with equimolar amounts of the sulfonamide salt of the formula (IIa). Here, the sulfonamide salt of the formula (IIa) can be employed directly or formed in situ—for example by reacting the corresponding sulfonamide with a suitable base M⁺X⁻, where M⁺ is an ammonium ion and X⁻ is, for example, a hydroxy or alkoxy anion.

7. Reaction of the neutral sulfonylurea (XX) with a primary, secondary or tertiary amine NRR′R″ (Scheme 17), where R, R′ and R″ are identical or different radicals selected from the group consisting of:
(a) H,
(b) an ammonium ion in which optionally one, two, three or all four hydrogen atoms are substituted by identical or different radicals from the group consisting of (C₁-C₄)-alkyl, hydroxy-(C₁-C₄)-alkyl, (C₃-C₆)-cycloalkyl, (C₁-C₄)-alkoxy-(C₁-C₄)-alkyl, hydroxy-(C₁-C₄)-alkoxy-(C₁-C₄)-alkyl, (C₁-C₆)-mercaptoalkyl, phenyl or benzyl, where the radicals mentioned above are optionally substituted by one or more identical or different radicals from the group consisting of halogen, such as F, Cl, Br or I, nitro, cyano, azido, (C₁-C₆)-alkyl, (C₁-C₆)-haloalkyl, (C₃-C₆)-cycloalkyl, (C₁-C₆)-alkoxy, (C₁-C₆)-haloalkoxy and phenyl, and where in each case two substituents at the nitrogen atom together optionally form an unsubstituted or substituted ring, or
(c) a saturated or unsaturated/aromatic nitrogenous heterocyclic ionic compound which has 1-10 carbon atoms in the ring system and is optionally mono- or polycondensed and/or mono- or polysubstituted by (C₁-C₄)-alkyl.

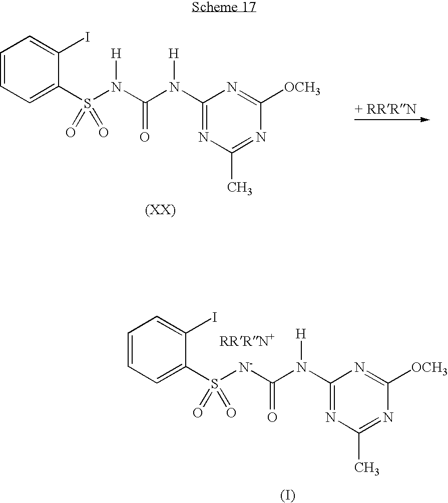

This reaction is carried out at temperatures between −20° C. and 100° C., preferably between −10° C. and 50° C., in inert solvents, such as, for example, tetrahydrofuran, methylene chloride or methanol, or mixtures of solvents.

The “inert solvents” referred to in the process variants above are in each case meant to be solvents which are inert under the reaction conditions in question, but which do not have to be inert under all reaction conditions.

Collections of salts according to the invention, in particular those of the formula (I), which can be synthesized by the reactions mentioned above, may also be prepared in a parallel manner, and this may be effected manually or in a semiautomated or fully automated manner. In this case, it is possible, for example, to automate the procedure of the reaction, the work-up or the purification of the products or of the intermediates. In total, this is to be understood as meaning a procedure as is described, for example, by S. H. DeWitt in “Annual Reports in Combinatorial Chemistry and Molecular Diversity: Automated Synthesis”, volume 1, Verlag Escom 1997, pages 69 to 77.

For carrying out microwave-supported syntheses, it is possible to use a microwave apparatus, for example the “Discover” model from CEM GmbH Mikrowellen-Analysentechnik, Carl-Friedrich-Gauβ-Str. 9, 47475 Kamp-Lintfort, Germany.

A number of commercially available apparatuses as they are offered by, for example, Stem Corporation, Woodrolfe Road, Tollesbury, Essex, England, H+P Labortechnik GmbH, Bruckmannring 28, 85764 Oberschleiβheim, Germany or Radleys, Shirehill, Saffron Walden, Essex, CB 11 3AZ, England, may be used for the parallel procedure of the reaction and work-up. For the parallel purification of compounds of the general formula (I) or of intermediates obtained during the preparation, use may be made, inter alia, of chromatography apparatuses, for example those from ISCO, Inc., 4700 Superior Street, Lincoln, Nebr. 68504, USA.

The apparatuses mentioned lead to a modular procedure in which the individual process steps are automated, but manual operations have to be performed between the process steps. This can be avoided by employing semi-integrated or fully integrated automation systems where the automation modules in question are operated by, for example, robots. Such automation systems can be obtained, for example, from Zymark Corporation, Zymark Center, Hopkinton, Mass. 01748, USA.

In addition to the methods described here, the salts according to the invention, in particular of compounds of the general formula (I), may be prepared fully or in part by solid-phase-supported methods. For this purpose, individual intermediates or all intermediates of the synthesis or a synthesis adapted to suit the procedure in question are bound to a synthetic resin. Solid-phase-supported synthesis methods are described extensively in the specialist literature, for example Barry A. Bunin in “The Combinatorial Index”, Academic Press, 1998.

The use of solid-phase-supported synthesis methods permits a number of protocols, which are known from the literature and which for their part may be performed manually or in an automated manner, to be carried out. For example, the “teabag method” (Houghten, U.S. Pat. No. 4,631,211; Houghten et al., Proc. Natl. Acad. Sci, 1985, 82, 5131-5135) in which products from IRORI, 11149 North Torrey Pines Road, La Jolla, Calif. 92037, USA, are employed, may be semiautomated. The automation of solid-phase-supported parallel syntheses is performed successfully, for example, by apparatures from Argonaut Technologies, Inc., 887 Industrial Road, San Carlos, Calif. 94070, USA or MultiSynTech GmbH, Wullener Feld 4, 58454 Witten, Germany.

The preparation according to the processes described herein affords the salts according to the invention, in particular compounds the formula (I), in the form of collections of substances referred to as libraries. The present invention also provides libraries which comprise at least two compounds according to the invention, in particular compounds of the formula (I).

The salts according to the invention, in particular the compounds of the formula (I), have excellent herbicidal activity against a broad spectrum of economically important monocotyledonous and dicotyledonous harmful plants. The active compounds also act efficiently on perennial weeds which produce shoots from rhizomes, rootstocks and other perennial organs and which are difficult to control. In this context, it is immaterial whether the substances are applied by the pre-sowing method, the pre-emergence method or the post-emergence method.

If the salts according to the invention, in particular the compounds of the formula (I), are applied to the soil surface prior to germination, then the weed seedlings are either prevented completely from emerging, or the weeds grow until they have reached the cotyledon stage but then their growth stops and, eventually, after three to four weeks have elapsed, they die completely.

If the active compounds are applied post-emergence to the green parts of the plants, growth also stops drastically a very short time after the treatment and the weed plants remain at the development stage of the point in time of application, or they die completely after a certain time, so that in this manner competition by the weeds, which is harmful to the crop plants, is eliminated at a very early point in time and in a sustained manner.

Although the salts according to the invention, in particular the compounds of the formula (I), have excellent herbicidal activity against monocotyledonous and dicotyledonous weeds, crop plants of economically important crops, such as, for example, wheat, barley, rye, oats, rice, corn, sugar cane, flax and other plantation crops, are not damaged at all, or only to a negligible extent. For these reasons, the present compounds are highly suitable for selectively controlling unwanted vegetation in agriculturally useful crops.

Furthermore, the salts according to the invention, in particular the compounds of the formula (I), have very advantageous properties with respect to their behavior in the environment, in particular with respect to their rotational behavior, i.e. to crops which are otherwise sensitive toward the compounds of the formula (I) according to the invention, such as, for example, sugar beet, sunflower or cruciferous plants, such as, for example, oilseed rape, mustard and wild turnip.

In addition, the salts according to the invention, in particular the compounds of the formula (I), have outstanding growth-regulating properties in crop plants. They engage in the plant metabolism in a regulating manner and can thus be employed for the targeted control of plant constituents and for facilitating harvesting, such as, for example, by provoking desiccation and stunted growth. Furthermore, they are also suitable for generally regulating and inhibiting undesirable vegetative growth, without destroying the plants in the process. Inhibition of vegetative growth plays an important role in many monocotyledonous and dicotyledonous crops because lodging can be reduced hereby, or prevented completely.

By virtue of their herbicidal and plant growth-regulatory properties, the active compounds can also be used for controlling harmful plants in crops of known or still to be developed genetically engineered plants. The transgenic plants generally have particularly advantageous properties, for example resistance to certain pesticides, in particular certain herbicides, resistance to plant diseases or causative organisms of plant diseases, such as certain insects or microorganisms, such as fungi, bacteria or viruses. Other particular properties relate, for example, to the quantity, quality, storage-stability, composition and to specific ingredients of the harvested product.

Thus, transgenic plants having an increased starch content or a modified quality of the starch or those having a different fatty acid composition of the harvested product are known. Likewise, by virtue of their herbicidal and plant growth-regulatory properties, the active compounds can also be used for controlling harmful plants in crops of known or still to be developed plants obtained by mutant selection.

The use of the salts according to the invention, in particular of compounds of the formula (I), in economically important transgenic crops or crops obtained by mutant selection of useful and ornamental plants, for example of cereals, such as wheat, barley, rye, oats, millet, rice, manioc and corn, or else in crops of oilseed rape, potato, tomato, pea and other vegetable species, is preferred.

Preferably, the salts according to the invention, in particular the compounds of the formula (I), can be used as herbicides in crops of useful plants which are resistant or which have been made resistant by genetic engineering toward the phytotoxic effects of the herbicides, or have been obtained by mutant selection. The salts according to the invention, in particular the compounds of the formula (I), may likewise preferably be used as herbicides in crops of useful plants which are a crossbreed of plants which have been made resistant by genetic engineering and plants which have been obtained by mutant selection, as described, for example, in WO 2007/024782.

Conventional ways of preparing novel plants which have modified properties compared to known plants comprise, for example, traditional breeding methods and the generation of mutants.

Alternatively, novel plants having modified properties can be generated with the aid of genetic engineering methods (see, for example, EP-A-0221044, EP-A-0131624). For example, there have been described several cases of
genetically engineered changes in crop plants in order to modify the starch synthesized in the plants (for example WO 92/11376, WO 92/14827, WO 91/19806),
transgenic crop plants which are resistant to certain herbicides of the glufosinate type (cf., for example, EP-A-0242236, EP-A-242246) or the glyphosate type (WO 92/00377) or the sulfonylurea type (EP-A-0257993, U.S. Pat. No. 5,013,659),
transgenic crop plants, for example cotton, having the ability to produce Bacillus thuringiensis toxins (Bt toxins) which impart resistance to certain pests to the plants (EP-A-0142924, EP-A-0193259), transgenic crop plants having a modified fatty acid composition (WO 91/13972).

Numerous molecular biological techniques which allow the preparation of novel transgenic plants having modified properties are known in principle; see, for example, Sambrook et al., 1989, Molecular Cloning, A Laboratory Manual, 2nd ed. Cold Spring Harbor Laboratory Press, Cold Spring Harbor, N.Y.; or Winnacker “Gene and Klone” [Genes and Clones], VCH Weinheim, 2nd edition 1996, or Christou, “Trends in Plant Science” 1 (1996) 423-431).

In order to carry out such genetic engineering manipulations, it is possible to introduce nucleic acid molecules into plasmids which allow a mutagenesis or a change in the sequence to occur by recombination of DNA sequences. Using the abovementioned standard processes it is possible, for example, to exchange bases, to remove partial sequences or to add natural or synthetic sequences. To link the DNA fragments with each other, it is possible to attach adaptors or linkers to the fragments.

Plant cells having a reduced activity of a gene product can be prepared, for example, by expressing at least one appropriate antisense-RNA, a sense-RNA to achieve a cosuppression effect, or by expressing at least one appropriately constructed ribozyme which specifically cleaves transcripts of the abovementioned gene product. To this end, it is possible to employ both DNA molecules which comprise the entire coding sequence of a gene product including any flanking sequences that may be present, and DNA molecules which comprise only parts of the coding sequence, it being necessary for these parts to be long enough to cause an antisense effect in the cells. It is also possible to use DNA sequences which have a high degree of homology to the coding sequences of a gene product but which are not entirely identical. When expressing nucleic acid molecules in plants, the synthesized protein can be localized in any desired compartment of the plant cell. However, to achieve localization in a certain compartment, it is, for example, possible to link the coding region with DNA sequences which ensure localization in a certain compartment. Such sequences are known to the person skilled in the art (see, for example, Braun et al., EMBO J. 11 (1992), 3219-3227; Wolter et al., Proc. Natl. Acad. Sci. USA 85 (1988), 846-850; Sonnewald et al., Plant J. 1 (1991), 95-106).

The transgenic plant cells can be regenerated to whole plants using known techniques. The transgenic plants can in principle be plants of any desired plant species, i.e. both monocotyledonous and dicotyledonous plants.

In this manner, it is possible to obtain transgenic plants which have modified properties by overexpression, suppression or inhibition of homologous (=natural) genes or gene sequences or by expression of heterologous (=foreign) genes or gene sequences.

The salts according to the invention, in particular compounds of the formula (I), can preferably be used in transgenic crops or crops obtained by mutant selection or crossbreeds/hybrids thereof which are resistant to herbicides selected from the group consisting of the sulfonylureas, glufosinate-ammonium or glyphosate-isopropylammonium and analogous active compounds.

When using the active compounds according to the invention in transgenic crops or crops obtained by mutant selection or crossbreeds thereof, in addition to the effects against harmful plants which can be observed in other crops, there are frequently effects which are specific for the application in the respective transgenic crops or crops obtained by mutant selection or crossbreeds thereof, for example a modified or specifically broadened spectrum of weeds which can be controlled, modified application rates which can be used for the application, preferably good combinability with the herbicides to which the transgenic crops are resistant, and an effect on the growth and the yield of the transgenic crop plants.

The invention therefore also provides for the use of the salts according to the invention, in particular of compounds of the formula (I), as herbicides for controlling harmful plants in transgenic crop plants or crop plants obtained by mutation selection or crossbreeds thereof.

The compounds according to the invention can be applied in various customary formulations, for example in the form of wettable powders, emulsifiable concentrates, sprayable solutions, dusts or granules. The invention therefore also provides herbicidal and plant-growth-regulating compositions comprising the compounds of the formula (I).

The salts according to the invention, in particular the compounds of the formula (I), can be formulated in various ways depending on the prevailing biological and/or chemico-physical parameters. Examples of suitable formulation options are: wettable powders (WP), water-soluble powders (SP), water-soluble concentrates, emulsifiable concentrates (EC), emulsions (EW), such as oil-in-water and water-in-oil emulsions, sprayable solutions, suspension concentrates (SC), oil- or water-based dispersions, oil-miscible solutions, capsule suspensions (CS), dusts (DP), seed-dressing compositions, granules for broadcasting and soil application, granules (GR) in the form of microgranules, spray granules, coating granules and adsorption granules, water-dispersible granules (WG), water-soluble granules (SG), ULV formulations, microcapsules and waxes.

These individual formulation types are known in principle and are described, for example, in Winnacker-Küchler, “Chemische Technologie” [Chemical Technology], Volume 7, C. Hauser Verlag Munich, 4th edition 1986; Wade van Valkenburg, “Pesticide Formulations”, Marcel Dekker, N.Y., 1973; K. Martens, “Spray Drying” Handbook, 3rd ed. 1979, G. Goodwin Ltd. London.

The necessary formulation auxiliaries, such as inert materials, surfactants, solvents and other additives, are likewise known and are described, for example, in: Watkins, “Handbook of Insecticide Dust Diluents and Carriers”, 2nd ed., Darland Books, Caldwell N.J., H. v. Olphen, “Introduction to Clay Colloid Chemistry”; 2nd ed., J. Wiley & Sons, N.Y.; C. Marsden, “Solvents Guide”; 2nd ed., Interscience, N.Y. 1963; McCutcheon's “Detergents and Emulsifiers Annual”, MC Publ. Corp., Ridgewood N.J.; Sisley and Wood, “Encyclopedia of Surface Active Agents”, Chem. Publ. Co. Inc., N.Y. 1964; Schönfeldt, “Grenzflächenaktive Äthylenoxidaddukte” [Surface-active ethylene oxide adducts], Wiss. Verlagsgesell., Stuttgart 1976; Winnacker-Küchler, “Chemische Technologie” [Chemical Technology], Volume 7, C. Hauser Verlag Munich, 4th edition 1986.

Based on these formulations it is also possible to produce combinations with other pesticidally active substances, for example insecticides, acaricides, herbicides and fungicides, and also with safeners, fertilizers and/or growth regulators, for example in the form of a ready mix or tank mix.

Wettable powders are preparations which are uniformly dispersible in water and which contain, in addition to the active compound and as well as a diluent or inert substance, surfactants of ionic and/or nonionic type (wetting agents, dispersants), for example polyethoxylated alkyl phenols, polyethoxylated fatty alcohols, polyethoxylated fatty amines, fatty alcohol polyglycol ether sulfates, alkanesulfonates, alkylbenzenesulfonates, sodium lignosulfonate, sodium 2,2′-dinaphthylmethane-6,6′-disulfonate, sodium dibutylnaphthalenesulfonate or else sodium oleoylmethyltaurinate. To prepare the wettable powders, the herbicidally active compounds are finely ground, for example in customary apparatus such as hammer mills, fan mills and air-jet mills, and are mixed simultaneously or subsequently with the formulation auxiliaries.

Emulsifiable concentrates are prepared by dissolving the active compound in an organic solvent, for example butanol, cyclohexanone, dimethylformamide, xylene or else relatively high-boiling aromatics or hydrocarbons or mixtures of the organic solvents, with the addition of one or more surfactants of ionic and/or nonionic type (emulsifiers). Examples of emulsifiers which can be used are calcium alkylarylsulfonates, such as Ca dodecylbenzenesulfonate, or nonionic emulsifiers, such as fatty acid polyglycol esters, alkylaryl polyglycol ethers, fatty alcohol polyglycol ethers, propylene oxide-ethylene oxide condensation products, alkyl polyethers, sorbitan esters, for example sorbitan fatty acid esters or polyoxyethylene sorbitan esters, for example polyoxyethylene sorbitan fatty acid esters. Dusts are obtained by grinding the active compound with finely divided solid substances, for example talc, natural clays, such as kaolin, bentonite and pyrophyllite, or diatomaceous earth.

Suspension concentrates can be water- or oil-based. They can be prepared, for example, by wet milling using commercially customary bead mills, with or without the addition of surfactants as already mentioned above, for example, in the case of the other formulation types.

Emulsions, for example oil-in-water emulsions (EW), can be prepared for example by means of stirrers, colloid mills and/or static mixers using aqueous organic solvents and, if desired, surfactants as already mentioned above, for example, in the case of the other formulation types.

Granules can be prepared either by spraying the active compound onto adsorptive, granulated inert material or by applying active-compound concentrates to the surface of carriers such as sand, kaolinites or granulated inert material, by means of adhesive binders, for example polyvinyl alcohol, sodium polyacrylate or else mineral oils. Suitable active compounds can also be granulated in the manner which is customary for the preparation of fertilizer granules, if desired as a mixture with fertilizers.

Water-dispersible granules are generally prepared by the customary processes, such as spray-drying, fluidized-bed granulation, disk granulation, mixing using high-speed mixers, and extrusion without solid inert material.

For the preparation of disk, fluidized-bed, extruder and spray granules, see for example processes in “Spray-Drying Handbook” 3rd ed. 1979, G. Goodwin Ltd., London; J. E. Browning, “Agglomeration”, Chemical and Engineering 1967, pages 147 ff; “Perry's Chemical Engineer's Handbook”, 5th ed., McGraw-Hill, New York 1973, pp. 8-57.

For further details on the formulation of crop protection products, see for example G. C. Klingman, “Weed Control as a Science”, John Wiley and Sons., Inc., New York, 1961, pages 81-96 and J. D. Freyer, S. A. Evans, “Weed Control Handbook”, 5th ed., Blackwell Scientific Publications, Oxford, 1968, pages 101-103.

The agrochemical formulations generally contain from 0.1 to 99% by weight, preferably from 0.1 to 95% by weight, particularly preferably from 0.5 to 90% by weight, of the salts according to the invention, in particular the compounds of the formula (I).

In wettable powders the concentration of active compound is, for example, from about 10 to 90% by weight, the remainder to 100% by weight consisting of customary formulation constituents. In emulsifiable concentrates the concentration of active compound can be from about 1 to 90%, preferably from 5 to 80%, by weight. Formulations in the form of dusts contain from 1 to 30% by weight of active compound, preferably most commonly from 5 to 20% by weight of active compound, while sprayable solutions contain from about 0.05 to 80%, preferably from 2 to 50%, by weight of active compound. In the case of water-dispersible granules, the content of active compound depends partly on whether the active compound is in liquid or solid form and on the granulation auxiliaries, fillers, etc. that are used. In water-dispersible granules the content of active compound, for example, is between 1 and 95% by weight, preferably between 10 and 80% by weight.

In addition, said formulations of active compound may comprise the tackifiers, wetting agents, dispersants, emulsifiers, penetrants, preservatives, antifreeze agents, solvents, fillers, carriers, colorants, antifoams, evaporation inhibitors and pH and viscosity regulators which are customary in each case.

If the extender used is water, it is also possible to use, for example, organic solvents as auxiliary solvents. Suitable liquid solvents are essentially: aromatics, such as xylene, toluene, alkylnaphthalenes, chlorinated aromatics or chlorinated aliphatic hydrocarbons, such as chlorobenzenes, chloroethylenes, or methylene chloride, aliphatic hydrocarbons, such as cyclohexane or paraffins, for example mineral oil fractions, mineral and vegetable oils, alcohols, such as butanol or glycol, and ethers and esters thereof, ketones, such as acetone, methyl ethyl ketone, methyl isobutyl ketone or cyclohexanone, strongly polar solvents, such as dimethylformamide or dimethyl sulfoxide, and also water.

Suitable solid carriers are: for example ammonium salts and ground natural minerals, such as kaolins, clays, talc, chalk, quartz, attapulgite, montmorillonite or diatomaceous earth, and ground synthetic minerals, such as finely divided silica, alumina and silicates; suitable solid carriers for granules are: for example crushed and fractionated natural rocks, such as calcite, marble, pumice, sepiolite and dolomite, and also synthetic granules of inorganic and organic meals, and granules of organic material, such as sawdust, coconut shells, corn cobs and tobacco stalks; suitable emulsifiers and/or foam formers are: for example nonionic and anionic emulsifiers, such as polyoxyethylene fatty acid esters, polyoxyethylene fatty alcohol ethers, for example alkylaryl polyglycol ethers, alkylsulfonates, alkyl sulfates, arylsulfonates and also protein hydrolyzates; suitable dispersants are: for example lignosulfite waste liquors and methylcellulose.

Tackifiers such as carboxymethylcellulose and natural and synthetic polymers in the form of powders, granules or latices, such as gum arabic, polyvinyl alcohol and polyvinyl acetate, and also natural phospholipids, such as cephalins and lecithins, and synthetic phospholipids, can be used in the formulations.

The herbicidal action of the herbicide combinations according to the invention can be improved, for example, by surfactants, preferably by wetting agents from the group of the fatty alcohol polyglycol ethers. The fatty alcohol polyglycol ethers preferably comprise 10-18 carbon atoms in the fatty alcohol radical and 2-20 ethylene oxide units in the polyglycol ether moiety. The fatty alcohol polyglycol ethers may be present in nonionic form, or ionic form, for example in the form of fatty alcohol polyglycol ether sulfates, which may be used, for example, as alkali metal salts (for example sodium salts and potassium salts) or ammonium salts, or even as alkaline earth metal salts, such as magnesium salts, such as Cᵤ/Cᵤ-fatty alcohol diglycol ether sulfate sodium (Genapol® LRO, Clariant GmbH); see, for example, EP-A-0476555, EP-A-0048436, EP-A-0336151 or U.S. Pat. No. 4,400,196 and also Proc. EWRS Symp. “Factors Affecting Herbicidal Activity and Selectivity”, 227-232 (1988). Nonionic fatty alcohol polyglycol ethers are, for example, (C₁₀-C₁₈)-, preferably (C₁₀-C₁₄)-fatty alcohol polyglycol ethers (for example isotridecyl alcohol polyglycol ethers) which comprise, for example, 2-20, preferably 3-15, ethylene oxide units, for example those from the Genapol® X-series, such as Genapol® X-030, Genapol® X-060, Genapol® X-080 or Genapol® X-150 (all from Clariant GmbH).

The present invention further comprises the combination of components A and B with the wetting agents mentioned above from the group of the fatty alcohol polyglycol ethers which preferably contain 10-18 carbon atoms in the fatty alcohol radical and 2-20 ethylene oxide units in the polyglycol ether moiety and which may be present in nonionic or ionic form (for example as fatty alcohol polyglycol ether sulfates). Preference is given to Cᵤ/Cᵤ-fatty alcohol diglycol ether sulfate sodium (Genapol® LRO, Clariant GmbH) and isotridecyl alcohol polyglycol ether having 3-15 ethylene oxide units, for example from the Genapol® X-series, such as Genapol® X-030, Genapol® X-060, Genapol® X-080 and Genapol® X-150 (all from Clariant GmbH). Furthermore, it is known that fatty alcohol polyglycol ethers, such as nonionic or ionic fatty alcohol polyglycol ethers (for example fatty alcohol polyglycol ether sulfates) are also suitable for use as penetrants and activity enhancers for a number of other herbicides, inter alia for herbicides from the group of the imidazolinones (see for example EP-A-0502014).

Furthermore, it is known that fatty alcohol polyglycol ethers, such as nonionic or ionic fatty alcohol polyglycol ethers (for example fatty alcohol polyglycol ether sulfates) are also suitable for use as penetrants and activity enhancers for a number of other herbicides, inter alia for herbicides from the group of the imidazolinones (see for example EP-A-0502014).

The herbicidal action of the herbicide combinations according to the invention can also be enhanced by using vegetable oils. The term vegetable oils is to be understood as meaning oils of oleaginous plant species, such as soybean oil, rapeseed oil, corn oil, sunflower oil, cottonseed oil, linseed oil, coconut oil, palm oil, thistle oil or castor oil, in particular rapeseed oil, and also their transesterification products, for example alkyl esters, such as rapeseed oil methyl ester or rapeseed oil ethyl ester.

The vegetable oils are preferably esters of C₁₀-C₂₂-, preferably C₁₂-C₂₀-, fatty acids. The C₁₀-C₂₂-fatty acid esters are, for example, esters of unsaturated or saturated C₁₀-C₂₂-fatty acids, in particular those having an even number of carbon atoms, for example erucic acid, lauric acid, palmitic acid and in particular C₁₈-fatty acids, such as stearic acid, oleic acid, linoleic acid or linolenic acid.

Examples of C₁₀-C₂₂-fatty acid esters are esters obtained by reacting glycerol or glycol with the C₁₀-C₂₂-fatty acids contained, for example, in oils of oleaginous plant species, or C₁-C₂₀-alkyl-C₁₀-C₂₂-fatty acid esters which can be obtained, for example, by transesterification of the abovementioned glycerol- or glycol-C₁₀-C₂₂-fatty acid esters with C₁-C₂₀-alcohols (for example methanol, ethanol, propanol or butanol). The transesterification can be carried out by known methods as described, for example, in Römpp Chemie Lexikon, 9th edition, Volume 2, page 1343, Thieme Verlag Stuttgart.

Preferred C₁-C₂₀-alkyl-C₁₀-C₂₂-fatty acid esters are methyl esters, ethyl esters, propyl esters, butyl esters, 2-ethylhexyl esters and dodecyl esters. Preferred glycol- and glycerol-C₁₀-C₂₂-fatty acid esters are the uniform or mixed glycol esters and glycerol esters of C₁₀-C₂₂-fatty acids, in particular fatty acids having an even number of carbon atoms, for example erucic acid, lauric acid, palmitic acid and, in particular, C₁₈-fatty acids, such as stearic acid, oleic acid, linoleic acid or linolenic acid.

In the herbicidal compositions according to the invention, the vegetable oils can be present, for example, in the form of commercially available oil-containing formulation additives, in particular those based on rapeseed oil, such as Hasten® (Victorian Chemical Company, Australia, hereinbelow referred to as Hasten, main ingredient: rapeseed oil ethyl ester), Actirob®B (Novance, France, hereinbelow referred to as ActirobB, main ingredient: rapeseed oil methyl ester), Rako-Binol® (Bayer AG, Germany, hereinbelow referred to as Rako-Binol, main ingredient: rapeseed oil), Renol® (Stefes, Germany, hereinbelow referred to as Renol, vegetable oil ingredient: rapeseed oil methyl ester) or Stefes Mero® (Stefes, Germany, hereinbelow referred to as Mero, main ingredient: rapeseed oil methyl ester).

In a further embodiment, the present invention comprises combinations with the vegetable oils mentioned above, such as rapeseed oil, preferably in the form of commercially available oil-containing formulation additives, in particular those based on rapeseed oil, such as Hasten® (Victorian Chemical Company, Australia, hereinbelow referred to as Hasten, main ingredient: rapeseed oil ethyl ester), Actirob®B (Novance, France, hereinbelow referred to as ActirobB, main ingredient: rapeseed oil methyl ester), Rako-Binol® (Bayer AG, Germany, hereinbelow referred to as Rako-Binol, main ingredient: rapeseed oil), Renol® (Stefes, Germany, hereinbelow referred to as Renol, vegetable oil ingredient: rapeseed oil methyl ester) or Stefes Mero® (Stefes, Germany, hereinbelow referred to as Mero, main ingredient: rapeseed oil methyl ester).

It is possible to use colorants, such as inorganic pigments, for example iron oxide, titanium oxide, Prussian Blue, and organic dyes, such as alizarin dyes, azo dyes and metal phthalocyanine dyes, and trace nutrients such as salts of iron, manganese, boron, copper, cobalt, molybdenum and zinc.

The salts according to the invention, in particular the compounds of the formula (I), can be employed as such or in the form of their preparations (formulations) combined with other pesticidally active compounds, such as, for example, insecticides, acaricides, nematicides, herbicides, fungicides, safeners, fertilizers and/or growth regulators, for example as finish formulation or as tank mixes.

Suitable as combination partners for the salts according to the invention, in particular for the compounds of the formula (I) in formulations of mixtures or in tank-mixes are, for example, known, preferably herbicidally active compounds whose action is based on the inhibition of, for example, acetolactate synthase, acetyl-coenzyme-A carboxylase, PS I, PS II, HPPDO, phytoene desaturase, protoporphyrinogen oxidase, glutamine synthetase, cellulose biosynthesis, 5-enolpyruvylshikimate 3-phosphate synthetase. Such compounds and also other compounds which can be used, in some cases having an unknown or a different mechanism of action, are described, for example, in Weed Research 26, 441-445 (1986), or in “The Pesticide Manual”, 12th edition 2000, or 13th edition 2003 or 14h edition 2006/2007, or in the corresponding “e-Pesticide Manual”, version 4 (2006), all published by the British Crop Protection Council, (hereinbelow also referred to in short as “PM”), and in the literature cited therein. Lists of “common names” are also available in “The Compendium of Pesticide Common Names” on the Internet. Examples of herbicides known from the literature which may be combined with the compounds of the formula (I) are, for example, the following active compounds (note: the herbicides are referred to either by the “common name” according to the International Organization for Standardization (ISO) or by the chemical name, if appropriate together with a customary code number and in each case comprise all use forms, such as acids, salts, esters and isomers, such as stereoisomers and optical isomers, in particular the commercial form or the commercial forms, unless the context indicates otherwise. In the case of sulfonamides such as sulfonylureas, salts also include salts formed by exchange of a hydrogen atom at the sulfonamide group for a cation. Here one and, in some cases, more application forms are mentioned):

acetochlor; acibenzolar-S-methyl; acifluorfen(-sodium); aclonifen; AD-67; AKH 7088, i.e. [[[1-[5-[2-chloro-4-(trifluoromethyl)phenoxy]-2-nitrophenyl]-2-methoxyethylidene]-amino]oxy]acetic acid and methyl [[[1-[5-[2-chloro-4-(trifluoromethyl)phenoxy]-2-nitrophenyl]-2-methoxyethylidene]amino]oxy]acetate; alachlor; alloxydim(-sodium); ametryn; amicarbazone, amidochlor, amidosulfuron; aminopyralid; amitrol; ammonium pelargonate; AMS, i.e. ammonium sulfamat; ancimidol; anilofos; asulam; atrazine; aviglycine; azafenidin, azimsulfuron (DPX-A8947); aziprotryn; barban; BAS 516 H, i.e. 5-fluoro-2-phenyl-4H-3,1-benzoxazin-4-one; beflubutamid (UBH-509), benazolin(-ethyl); bencarbazone; benfluralin; benfuresate; bensulfuron(-methyl); bensulide; bentazone; benzfendizone; benzobicyclon, benzofenap; benzofluor; benzoylprop(-ethyl); benzthiazuron; bialaphos; bifenox; bispyribac(-sodium) (KIN-2023); borax; bromacil; bromobutide; bromofenoxim; bromoxynil; bromuron; buminafos; busoxinone; butachlor; butafenacil; butamifos; butenachlor (KH-218); buthidazole; butralin; butroxydim; butylate; cafenstrole (CH-900); caloxydim; carbetamide; carfentrazone(-ethyl); catechin; CDAA, i.e. 2-chloro-N,N-di-2-propenylacetamid; CDEC, i.e. 2-chloroallyl diethyldithiocarbamate; chlormesulon; chlomethoxyfen; chloramben; chlorazifop-butyl; chlorbromuron; chlorbufam; chlorfenac; chlorfenprop; chlorflurecol(-methyl); chlorflurenol(-methyl); chloridazon; chlorimuron(-ethyl); chlormequat(-chloride); chlornitrofen; chlorophthalim (MK-616); chlorotoluron; chloroxuron; chlorpropham; chlorsulfuron; chlorthal-dimethyl; chlorthiamid; chlortoluron; cinidon(-methyl and -ethyl); cinmethylin; cinosulfuron; clefoxydim; clethodim; clodinafop and its ester derivatives (for example clodinafop-propargyl); clofencet; clomazone; clomeprop; cloprop; cloproxydim; clopyralid; clopyrasulfuron(-methyl); cloransulam(-methyl); cumyluron (JC 940); cyanamide; cyanazine; cycloate; cyclosulfamuron (AC 104); cycloxydim; cycluron; cyhalofop and its ester derivatives (for example the butyl ester, DEH-112); cyperquat; cyprazine; cyprazole; daimuron; 2,4-D; 2,4-DB; dalapon; daminozide; dazomet; n-decanol; desmedipham; desmetryn; di-allate; dicamba; dichlobenil; dichlormid; dichlorprop(-P) salts; diclofop and its esters, such as diclofop-methyl; diclofop-P(-methyl); diclosulam; diethatyl(-ethyl); difenoxuron; difenzoquat(metilsulfate); diflufenican; diflufenzopyr(-sodium); dimefuron; dimepiperate; dimethachlor; dimethametryn; dimethazone; dimethenamid (SAN-582H); dimethenamide-P; dimethylarsinic acid; dimethipin; dimetrasulfuron; dimexyflam; dinitramine; dinoseb; dinoterb; diphenamid; dipropetryn; diquat salts; dithiopyr; diuron; DNOC; eglinazine-ethyl; EL 77, i.e. 5-cyano-1-(1,1-dimethylethyl)-N-methyl-1H-pyrazole-4-carboxamide; endothal; epoprodan; EPTC; esprocarb; ethalfluralin; ethametsulfuron-methyl; ethephon; ethidimuron; ethiozin; ethofumesate; ethoxyfen and its esters (for example ethyl ester, HN-252); ethoxysulfuron; etobenzanid (HW 52); F5231, i.e. N-[2-chloro-4-fluoro-5-[4-(3-fluoropropyl)-4,5-dihydro-5-oxo-1H-tetrazol-1-yl]phenyl]ethanesulfonamide; fenchlorazole(-ethyl); fenclorim; fenoprop; fenoxan, fenoxapropand fenoxaprop-P and also their esters, for example fenoxaprop-P-ethyl and fenoxaprop-ethyl; fenoxydim; fentrazamide; fenuron; ferrous sulfate; flamprop(-methyl or -isopropyl or -isopropyl-L); flamprop-M(-methyl or -isopropyl); flazasulfuron; floazulate (JV-485); florasulam; fluazifop and fluazifop-P and their esters, for example fluazifop-butyl and fluazifop-P-butyl; fluazolate; flucarbazone(-sodium); flucetosulfuron; fluchloralin; flufenacet; flufenpyr(-ethyl); flumetralin; flumetsulam; flumeturon; flumiclorac(-pentyl); flumioxazin (S-482); flumipropyn; fluometuron; fluorochloridone; fluorodifen; fluoroglycofen(-ethyl); flupoxam (KNW-739); flupropacil (UBIC-4243); flupropanoate; flupyrsulfuron(-methyl)(-sodium); flurenol(-butyl); fluridone; fluorochloridone; fluoroxypyr(-meptyl); flurprimidol; flurtamone; fluthiacet(-methyl) (KIN-9201); fluthiamide; fluxofenim; fomesafen; foramsulfuron; forchlorfenuron; fosamine; furyloxyfen; gibberillic acid; glufosinate(-ammonium); glyphosate(-isopropylammonium); halosafen; halosulfuron(-methyl); haloxyfop and its esters; haloxyfop-P (=R-haloxyfop) and its esters; HC-252; hexazinone; HNPC-C9908, i.e. methyl 2-[[[[[4-methoxy-6-(methylthio)-2-pyrimidinyl]amino]carbonyl]amino]sulfonyl]benzoate; imazamethabenz(-methyl); imazamox; imazapic; imazapyr; imazaquin and salts, such as the ammonium salt; imazethapyr; imazosulfuron; inabenfide; indanofan; iodosulfuron-methyl(-sodium); ioxynil; isocarbamid; isopropalin; isoproturon; isouron; isoxaben; isoxachlortole; isoxaflutole; isoxapyrifop; karbutilate; lactofen; lenacil; linuron; maleic hyrazide (MH); MBTA; MCPA; MCPB; mecoprop(-P); mefenacet; mefluidide; mepiquat(-chloride); mesosulfuron(-methyl); mesotrione; metam; metamifop; metamitron; metazachlor; methabenzthiazuron; metham; methazole; methoxyphenone; methylarsonic acid; methylcyclopropene; methyldymron; methylisothiocyanate; methabenzthiazuron; metobenzuron; metobromuron; (alpha-)metolachlor; metosulam (XRD 511); metoxuron; metribuzin; metsulfuron-methyl; molinate; monalide; monocarbamide dihydrogensulfate; monolinuron; monuron; monosulfuron; MT 128, i.e. 6-chloro-N-(3-chloro-2-propenyl)-5-methyl-N-phenyl-3-pyridazinamine; MT 5950, i.e. N-[3-chloro-4-(1-methylethyl)-phenyl]-2-methylpentanamide; naproanilide; napropamide; naptalam; NC 310, i.e. 4-(2,4-dichlorobenzoyl)-1-methyl-5-benzyloxypyrazole; neburon; nicosulfuron; nipyraclofen; nitralin; nitrofen; nitrophenolate mixture; nitrofluorfen; nonanoic acid; norflurazon; orbencarb; orthosulfamuron; oxabetrinil; oryzalin; oxadiargyl (RP-020630); oxadiazon; oxasulfuron; oxaziclomefone; oxyfluorfen; paclobutrazol; paraquat(-dichloride); pebulate; pelargonic acid; pendimethalin; penoxulam; pentachlorophenol; pentanochlor; pentoxazone; perfluidone; pethoxamid; phenisopham; phenmedipham; picloram; picolinafen; pinoxaden; piperophos; piributicarb; pirifenop-butyl; pretilachlor; primisulfuron(-methyl); probenazole; procarbazone-(sodium); procyazine; prodiamine; profluralin; profoxydim; prohexadione(-calcium); prohydrojasmon; proglinazine(-ethyl); prometon; prometryn; propachlor; propanil; propaquizafop and its esters; propazine; propham; propisochlor; propoxycarbazone(-sodium) (MKH-6561); propyzamide; prosulfalin; prosulfocarb; prosulfuron (CGA-152005); prynachlor; pyraclonil; pyraflufen(-ethyl) (ET-751); pyrasulfotole; pyrazolynate; pyrazon; pyrazosulfuron(-ethyl); pyrazoxyfen; pyribambenz-isopropyl (ZJ 0702); pyrimbambenz-propyl (ZJ 0273); pyribenzoxim; pyributicarb; pyridafol; pyridate; pyriftalid; pyriminobac(-methyl) (KIN-6127); pyrimisulfan (KIN-5996); pyrithiobac(-sodium) (KIN-2031); pyroxasulfone (KIN-485); pyroxofop and its esters (for example propargyl ester); pyroxsulam; quinclorac; quinmerac; quinoclamine; quinofop and its ester derivatives, quizalofop and quizalofop-P and their ester derivatives, for example quizalofop-ethyl; quizalofop-P-tefuryl and -ethyl; renriduron; rimsulfuron (DPX-E 9636); S 275, i.e. 2-[4-chloro-2-fluoro-5-(2-propynyloxy)phenyl]-4,5,6,7-tetrahydro-2H-indazole; secbumeton; sethoxydim; siduron; simazine; simetryn; sintofen; SN 106279, i.e. 2-[[7-[2-chloro-4-(trifluoromethyl)phenoxy]-2-naphthalenyl]oxy]propanoic acid and methyl 2-[[7-[2-chloro-4-(trifluoromethyl)phenoxy]-2-naphthalenyl]oxy]propanoate; sulcotrione; sulfentrazone (FMC-97285, F-6285); sulfazuron; sulfometuron(-methyl); sulfosate (ICI-A0224); sulfosulfuron; TCA(-sodium); tebutam (GCP-5544); tebuthiuron; tecnacene; tefuryltrione; tembotrione; tepraloxydim; terbacil; terbucarb; terbuchlor; terbumeton; terbuthylazine; terbutryn; TFH 450, i.e. N,N-diethyl-3-[(2-ethyl-6-methylphenyl)sulfonyl]-1H-1,2,4-triazole-1-carboxamide; thenylchlor (NSK-850); thiafluamide, thiazafluoron; thiazopyr (Mon-13200); thidiazimin (SN-24085); thidiazuron; thiencarbazone(-methyl); thifensulfuron(-methyl); thiobencarb; Ti 35; tiocarbazil; topramezone; tralkoxydim; tri-allate; triasulfuron; triaziflam; triazofenamide; tribenuron(-methyl); triclopyr; tridiphane; trietazine; trifloxysulfuron(-sodium); trifluralin; triflusulfuron and esters (for example methyl ester, DPX-66037); trimeturon; trinexapac; tritosulfuron; tsitodef; uniconazole; vernolate; WL 110547, i.e. 5-phenoxy-1-[3-(trifluoromethyl)phenyl]-1H-tetrazole; D-489; ET-751; KIH-218; KIH-485; KIH-509; KPP-300; LS 82-556; NC-324; NC-330; DPX-N8189; SC-0774; DOWCO-535; DK-8910; V-53482; PP-600; MBH-001; TH-547; SYN-523; IDH-100; SYP-249; HOK-201; IR-6396; MTB-951; NC-620.

Of particular interest is the selective control of harmful plants in crops of useful plants and ornamental plants. In many crops, the salts according to the invention, in particular the compounds of the formula (I), already have very good to satisfactory selectivity; however, in principle, in some crops and especially also in the case of mixtures with other less selective herbicides, phytotoxicities on the crop plants may occur. In this respect, combinations of the salts according to the invention, in particular of compounds of the formula (I), which comprise the salts according to the invention, in particular the compounds of the formula (I), or combinations thereof with other herbicides or pesticides and safeners are of particular interest. The safeners, which are employed in antidotically active amounts, reduce the phytotoxic side effects of the herbicides/pesticides used, for example in economically important crops, such as cereals (wheat, barley, rye, corn, rice, millet), sugar beet, sugarcane, oilseed rape, cotton and soybeans, preferably cereals.

The safeners are preferably selected from the group consisting of:

A) compounds of the formula (S-I)

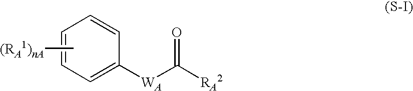

where the symbols and indices have the following meanings:
n_{A} is a natural number from 0 to 5, preferably from 0 to 3;
R_{A}¹ is halogen, (C₁-C₄)-alkyl, (C₁-C₄)-alkoxy, nitro or (C₁-C₄)-haloalkyl;
W_{A} is an unsubstituted or substituted divalent heterocyclic radical from the group consisting of partially unsaturated or aromatic five-membered heterocycles having 1 to 3 hetero ring atoms of the type N or O, where at least one nitrogen atom and at most one oxygen atom is present in the ring, preferably a radical from the group consisting of (W_{A}¹) to (W_{A}⁴),

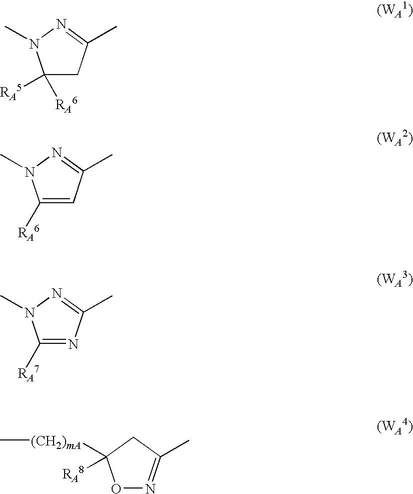

m_{A} is 0 or 1;
R_{A}² is OR_{A}³, SR_{A}³ or NR_{A}³R_{A}⁴ or a saturated or unsaturated 3- to 7-membered heterocycle having at least one nitrogen atom and up to 3 heteroatoms, preferably from the group consisting of O and S, which is attached via the nitrogen atom to the carbonyl group in (S-I) and which is unsubstituted or substituted by radicals from the group consisting of (C₁-C₄)-alkyl, (C₁-C₄)-alkoxy and optionally substituted phenyl, preferably a radical of the formula OR_{A}³, NHR_{A}⁴ or N(CH₃)₂, in particular of the formula OR_{A}³;
R_{A}³ is hydrogen or an unsubstituted or substituted aliphatic hydrocarbon radical having preferably a total of 1 to 18 carbon atoms;
R_{A}⁴ is hydrogen, (C₁-C₆)-alkyl, (C₁-C₆)-alkoxy or substituted or unsubstituted phenyl;
R_{A}⁵ is H, (C₁-C₈)-alkyl, (C₁-C₈)-haloalkyl), (C₁-C₄)-alkoxy-(C₁-C₈)-alkyl, cyano or COOR_{A}⁹ where R_{A}⁹ is hydrogen, (C₁-C₈)-alkyl, (C₁-C₈)-haloalkyl, (C₁-C₄)-alkoxy-(C₁-C₄)-alkyl, (C₁-C₆)-hydroxyalkyl, (C₃-C₁₂)-cycloalkyl or tri-(C₁-C₄)-alkylsilyl;
R_{A}⁶, R_{A}⁷, R_{A}⁸ are identical or different and are hydrogen, (C₁-C₈)-alkyl, (C₁-C₈)-haloalkyl, (C₃-C₁₂)-cycloalkyl or substituted or unsubstituted phenyl;
   preferably:
   a) compounds of the type of the dichlorophenylpyrazoline-3-carboxylic acid, preferably compounds such as ethyl 1-(2,4-dichlorophenyl)-5-(ethoxycarbonyl)-5-methyl-2-pyrazoline-3-carboxylate (S1-1) (“mefenpyr-diethyl”, see Pestic. Man.), and related compounds, as described in WO 91/07874;
   b) derivatives of dichlorophenylpyrazolecarboxylic acid, preferably compounds such as ethyl 1-(2,4-dichlorophenyl)-5-methylpyrazole-3-carboxylate (S1-2), ethyl 1-(2,4-dichlorophenyl)-5-isopropylpyrazole-3-carboxylate (S1-3), ethyl 1-(2,4-dichlorophenyl)-5-(1,1-dimethylethyl)pyrazole-3-carboxylate (S1-4), ethyl 1-(2,4-dichlorophenyl)-5-phenylpyrazole-3-carboxylate (S1-5) and related compounds, as described in EP-A-333 131 and EP-A-269 806;
   c) compounds of the type of the triazolecarboxylic acids, preferably compounds such as fenchlorazole(-ethyl ester), i.e. ethyl 1-(2,4-dichlorophenyl)-5-trichloro-methyl-(1H)-1,2,4-triazole-3-carboxylate (S1-6), and related compounds, as described in EP-A-174 562 and EP-A-346 620;
   d) compounds of the type of the 5-benzyl- or 5-phenyl-2-isoxazoline-3-carboxylic acid or the 5,5-diphenyl-2-isoxazoline-3-carboxylic acid, preferably compounds such as ethyl 5-(2,4-dichlorobenzyl)-2-isoxazoline-3-carboxylate (S1-7) or ethyl 5-phenyl-2-isoxazoline-3-carboxylate (S1-8) and related compounds, as described in WO 91/08202, or ethyl 5,5-diphenyl-2-isoxazolinecarboxylate (S1-9) (“isoxadifenethyl”) or n-propyl 5,5-diphenyl-2-isoxazolinecarboxylate (S1-10) or ethyl 5-(4-fluorophenyl)-5-phenyl-2-isoxazoline-3-carboxylate (S1-11), as described in the patent application WO-A-95/07897.
   B) Quinoline derivatives of the formula (S-II)

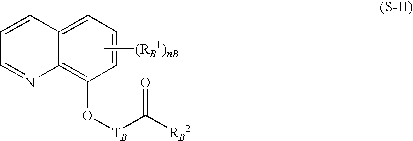

where the symbols and indices have the following meanings:
R_{B}¹ is halogen, (C₁-C₄)-alkyl, (C₁-C₄)-alkoxy, nitro or (C₁-C₄)-haloalkyl;
n_{B} is a natural number from 0 to 5, preferably from 0 to 3;
R_{B}² OR_{B}³, SR_{B}³ or NR_{B}³R_{B}⁴ or a saturated or unsaturated 3- to 7-membered heterocycle having at least one nitrogen atom and up to 3 heteroatoms, preferably from the group consisting of O and S, which is attached via the nitrogen atom to the carbonyl group in (S-II) and is unsubstituted or substituted by radicals from the group consisting of (C₁-C₄)-alkyl, (C₁-C₄)-alkoxy or optionally substituted phenyl, preferably a radical of the formula OR_{B}³, NHR_{B}⁴ or N(CH₃)₂, in particular of the formula OR_{B}³;
R_{B}³ is hydrogen or an unsubstituted or substituted aliphatic hydrocarbon radical having preferably a total of 1 to 18 carbon atoms;
R_{B}⁴ is hydrogen, (C₁-C₆)-alkyl, (C₁-C₆)-alkoxy or substituted or unsubstituted phenyl;
T_{B} is a (C₁- or C₂)-alkanediyl chain which is unsubstituted or substituted by one or two (C₁-C₄)-alkyl radicals or by [(C₁-C₃)-alkoxy]carbonyl;
preferably:
a) compounds of the type of the 8-quinolinoxyacetic acid (S2), preferably
1-methylhexyl (5-chloro-8-quinolinoxy)acetate (common name “cloquintocet-mexyl” (S2-1) (see Pestic. Man.),
1,3-dimethylbut-1-yl (5-chloro-8-quinolinoxy)acetate (S2-2),
4-allyloxybutyl (5-chloro-8-quinolinoxy)acetate (S2-3),
1-allyloxyprop-2-yl (5-chloro-8-quinolinoxy)acetate (S2-4),
ethyl (5-chloro-8-quinolinoxy)acetate (S2-5),
methyl (5-chloro-8-quinolinoxy)acetate (S2-6),
allyl (5-chloro-8-quinolinoxy)acetate (S2-7),
2-(2-propylideneiminoxy)-1-ethyl (5-chloro-8-quinolinoxy)acetate (S2-8), 2-oxoprop-1-yl (5-chloro-8-quinolinoxy)acetate (S2-9) and related compounds, as described in EP-A-86 750, EP-A-94 349 and EP-A-191 736 or EP-A-0 492 366, and also their hydrates and salts, as described in WO-A-2002/034048.
   b) Compounds of the type of the (5-chloro-8-quinolinoxy)malonic acid, preferably compounds such as diethyl (5-chloro-8-quinolinoxy)malonate, diallyl (5-chloro-8-quinolinoxy)malonate, methyl ethyl (5-chloro-8-quinolinoxy)malonate and related compounds, as described in EP-A-0 582 198.
   C) Compounds of the formula (S-III)

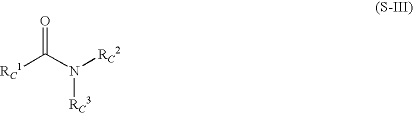

where the symbols and indices have the following meanings:
R_{C}¹ is (C₁-C₄)-alkyl, (C₁-C₄)-haloalkyl, (C₂-C₄)-alkenyl, (C₂-C₄)-haloalkenyl, (C₃-C₇)-cycloalkyl, preferably dichloromethyl;
R_{C}², R_{C}³ are identical or different and are hydrogen, (C₁-C₄)-alkyl, (C₂-C₄)-alkenyl, (C₂-C₄)-alkynyl, (C₁-C₄)-haloalkyl, (C₂-C₄)-haloalkenyl, (C₁-C₄)-alkylcarbamoyl-(C₁-C₄)-alkyl, (C₂-C₄)-alkenylcarbamoyl-(C₁-C₄)-alkyl, (C₁-C₄)-alkoxy-(C₁-C₄)-alkyl, dioxolanyl-(C₁-C₄)-alkyl, thiazolyl, furyl, furylalkyl, thienyl, piperidyl, substituted or unsubstituted phenyl, or R_{C}² and R_{C}³ together form a substituted or unsubstituted heterocyclic ring,
preferably an oxazolidine, thiazolidine, piperidine, morpholine, hexahydropyrimidine or benzoxazine ring;
preferably:
active compounds of the type of the dichloroacetamides which are frequently used as pre-emergence safener (soil-acting safeners), such as, for example,
“dichlormid” (see Pestic.Man.) (=N,N-diallyl-2,2-dichloroacetamide),
“R-29148” (=3-dichloroacetyl-2,2,5-trimethyl-1,3-oxazolidine from Stauffer),
“R-28725” (=3-dichloroacetyl-2,2,-dimethyl-1,3-oxazolidine from Stauffer),
“benoxacor” (see Pestic. Man.) (=4-dichloroacetyl-3,4-dihydro-3-methyl-2H-1,4-benzoxazine),
“PPG-1292” (=N-allyl-N-[(1,3-dioxolan-2-yl)methyl]dichloroacetamide from PPG Industries),
“DKA-24” (=N-allyl-N-[(allylaminocarbonyl)methyl]dichloroacetamide from Sagro-Chem),
“AD-67” or “MON 4660” (=3-dichloroacetyl-1-oxa-3-aza-spiro[4,5]decane from Nitrokemia or Monsanto),
“TI-35” (=1-dichloroacetylazepane from TRI-Chemical RT)
“diclonon” (dicyclonone) or “BAS145138” or “LAB145138” (=3-dichloroacetyl-2,5,5-trimethyl-1,3-diazabicyclo[4.3.0]nonane from BASF) and
“furilazole” or “MON 13900” (see Pestic. Man.) (=(RS)-3-dichloroacetyl-5-(2-furyl)-2,2-dimethyloxazolidine).
   D) N-Acylsulfonamides of the formula (S-IV) and their salts

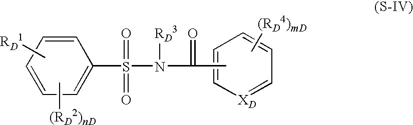

in which

### X_{D} is CH or N;

### R_{D}¹ is CO—NR_{D}⁵R_{D}⁶ or NHCO—R_{D}⁷;

R_{D}² is halogen, (C₁-C₄)-haloalkyl, (C₁-C₄)-haloalkoxy, nitro, (C₁-C₄)-alkyl, (C₁-C₄)-alkoxy, (C₁-C₄)-alkylsulfonyl, (C₁-C₄)-alkoxycarbonyl or (C₁-C₄)-alkylcarbonyl;
R_{D}³ is hydrogen, (C₁-C₄)-alkyl, (C₂-C₄)-alkenyl or (C₂-C₄)-alkynyl;
R_{D}⁴ is halogen, nitro, (C₁-C₄)-alkyl, (C₁-C₄)-haloalkyl, (C₁-C₄)-haloalkoxy, (C₃-C₆)-cycloalkyl, phenyl, (C₁-C₄)-alkoxy, cyano, (C₁-C₄)-alkylthio, (C₁-C₄)-alkylsulfinyl, (C₁-C₄)-alkylsulfonyl, (C₁-C₄)-alkoxycarbonyl or (C₁-C₄)-alkylcarbonyl;
R_{D}⁵ is hydrogen, (C₁-C₆)-alkyl, (C₃-C₆)-cycloalkyl, (C₂-C₆)-alkenyl, (C₂-C₆)-alkynyl, (C₅-C₆)-cycloalkenyl, phenyl or 3- to 6-membered heterocyclyl containing v_{D} heteroatoms from the group consisting of nitrogen, oxygen and sulfur, where the seven last-mentioned radicals are substituted by v_{D} substituents from the group consisting of halogen, (C₁-C₆)-alkoxy, (C₁-C₆)-haloalkoxy, (C₁-C₂)-alkylsulfinyl, (C₁-C₂)-alkylsulfonyl, (C₃-C₆)-cycloalkyl, (C₁-C₄)-alkoxycarbonyl, (C₁-C₄)-alkylcarbonyl and phenyl and, in the case of cyclic radicals, also (C₁-C₄)-alkyl and (C₁-C₄)-haloalkyl;
R_{D}⁶ is hydrogen, (C₁-C₆)-alkyl, (C₂-C₆)-alkenyl or (C₂-C₆)-alkynyl, where the three last-mentioned radicals are substituted by v_{D} radicals from the group consisting of halogen, hydroxy, (C₁-C₄)-alkyl, (C₁-C₄)-alkoxy and (C₁-C₄)-alkylthio, or
R_{D}⁵ and R_{D}⁶ together with the nitrogen atom carrying them form a pyrrolidinyl or piperidinyl radical;
R_{D}⁷ is hydrogen, (C₁-C₄)-alkylamino, di-(C₁-C₄)-alkylamino, (C₁-C₆)-alkyl, (C₃-C₆)-cycloalkyl, where the 2 last-mentioned radicals are substituted by v_{D} substituents from the group consisting of halogen, (C₁-C₄)-alkoxy, halogen-(C₁-C₆)-alkoxy and (C₁-C₄)-alkylthio and, in the case of cyclic radicals, also (C₁-C₄)-alkyl and (C₁-C₄)-haloalkyl;
n_{D} is 0, 1 or 2;
m_{D} is 1 or 2;
v_{D} is 0, 1, 2 or 3;
from among these, preference is given to compounds of the type of the N-acylsulfonamides, for example of the formula (S-V) below, which are known, for example, from WO 97/45016

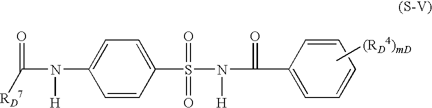

in which
R_{D}⁷ is (C₁-C₆)-alkyl, (C₃-C₆)-cycloalkyl, where the 2 last-mentioned radicals are substituted by v_{D} substituents from the group consisting of halogen, (C₁-C₄)-alkoxy, halogen-(C₁-C₆)-alkoxy and (C₁-C₄)-alkylthio and, in the case of cyclic radicals, also (C₁-C₄)-alkyl and (C₁-C₄)-haloalkyl;
R_{D}⁴ is halogen, (C₁-C₄)-alkyl, (C₁-C₄)-alkoxy, CF₃,
m_{D} is 1 or 2;
v_{D} is 0, 1, 2 or 3;
and also
acylsulfamoylbenzamides, for example of the formula (S-VI) below, which are known, for example, from WO 99/16744,

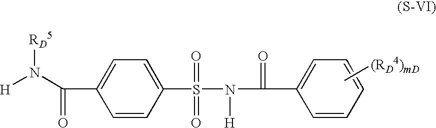

for example those in which
R_{D}⁵=cyclopropyl and (R_{D}⁴)=2-OMe (“cyprosulfamide”, S3-1),
R_{D}⁵=cyclopropyl and (R_{D}⁴)=5-Cl-2-OMe (S3-2),
R_{D}⁵=ethyl and (R_{D}⁴)=2-OMe (S3-3),
R_{D}⁵=isopropyl and (R_{D}⁴)=5-Cl-2-OMe (S3-4) and
R_{D}⁵=isopropyl and (R_{D}⁴)=2-OMe (S3-5);
and also
compounds of the type of the N-acylsulfamoylphenylureas of the formula (S-VII), which are known, for example, from EP-A-365484

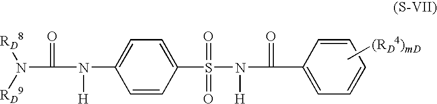

in which
R_{D}⁸ and R_{D}⁹ independently of one another are hydrogen, (C₁-C₈)-alkyl, (C₃-C₈)-cycloalkyl, (C₃-C₆)-alkenyl, (C₃-C₆)-alkynyl,
R_{D}⁴ is halogen, (C₁-C₄)-alkyl, (C₁-C₄)-alkoxy, CF₃
m_{D} is 1 or 2;
from among these in particular
1-[4-(N-2-methoxybenzoylsulfamoyl)phenyl]-3-methylurea,
1-[4-(N-2-methoxybenzoylsulfamoyl)phenyl]-3,3-dimethylurea,
1-[4-(N-4,5-dimethylbenzoylsulfamoyl)phenyl]-3-methylurea,
1-[4-(N-naphthoylsulfamoyl)phenyl]-3,3-dimethylurea,
   G) active compounds from the class of the hydroxyaromatics and aromatic-aliphatic carboxylic acid derivatives, for example
ethyl 3,4,5-triacetoxybenzoate, 3,5-dimethoxy-4-hydroxybenzoic acid, 3,5-dihydroxybenzoic acid, 4-hydroxysalicylic acid, 4-fluorosalicyclic acid, 1,2-dihydro-2-oxo-6-trifluoromethylpyridine-3-carboxamide, 2-hydroxycinnamic acid, 2,4-dichlorocinnamic acid, as described in WO 2004084631, WO 2005015994, WO 2006007981, WO 2005016001;
   H) active compounds from the class of the 1,2-dihydroquinoxalin-2-ones, for example
1-methyl-3-(2-thienyl)-1,2-dihydroquinoxalin-2-one, 1-methyl-3-(2-thienyl)-1,2-dihydroquinoxaline-2-thione, 1-(2-aminoethyl)-3-(2-thienyl)-1,2-dihydroquinoxalin-2-one hydrochloride, 1-(2-methylsulfonylaminoethyl)-3-(2-thienyl)-1,2-dihydroquinoxalin-2-one, as described in WO 2005112630,
   I) active compounds which, in addition to a herbicidal action against harmful plants, also have safener action on crop plants such as rice, such as, for example,
   “dimepiperate” or “MY-93” (see Pestic. Man.) (=S-1-methyl-1-phenylethyl piperidine-1-thiocarboxylate), which is known as safener for rice against damage by the herbicide molinate,
   “daimuron” or “SK 23” (see Pestic. Man.) (=1-(1-methyl-1-phenylethyl)-3-p-tolyl-urea), which is known as safener for rice against damage by the herbicide imazosulfuron,
   “cumyluron”=“JC-940” (=3-(2-chlorophenylmethyl)-1-(1-methyl-1-phenyl-ethyl)urea, see JP-A-60087254), which is known as safener for rice against damage by a number of herbicides,
   “methoxyphenone” or “NK 049” (=3,3′-dimethyl-4-methoxybenzophenone), which is known as safener for rice against damage by a number of herbicides,
   “CSB” (=1-bromo-4-(chloromethylsulfonyl)benzene) (CAS Reg. No. 54091-06-4 from Kumiai), which is known as safener against damage by a number of herbicides in rice,
   K) compounds of the formula (S-IX),
   as described in WO-A-1998/38856

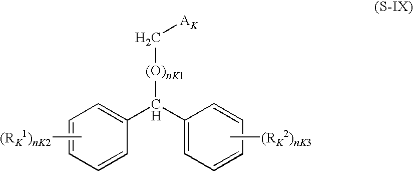

in which the symbols and indices have the following meanings:
R_{K}¹, R_{K}² independently of one another are halogen, (C₁-C₄)-alkyl, (C₁-C₄)-alkoxy, (C₁-C₄)-haloalkyl, (C₁-C₄)-alkylamino, di-(C₁-C₄)-alkylamino, nitro;

### A_{K} is COOR_{K}³ or COOR_{K}⁴

R_{K}³, R_{K}⁴ independently of one another are hydrogen, (C₁-C₄)-alkyl, (C₂-C₆)-alkenyl, (C₂-C₄)-alkynyl, cyanoalkyl, (C₁-C₄)-haloalkyl, phenyl, nitrophenyl, benzyl, halobenzyl, pyridinylalkyl or alkylammonium,
n_{K}¹ is 0 or 1,
n_{K}², n_{K}³ independently of one another are 0, 1 or 2
preferably: methyl (diphenylmethoxy)acetate (CAS Reg. No.: 41858-19-9),
L) compounds of the formula (S-X),
as described in WO A-98/27049

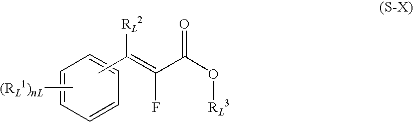

in which the symbols and indices have the following meanings:
X_{L} is CH or N,
n_{L} is, in the case that X=N, an integer from 0 to 4 and,
   in the case that X=CH, an integer from 0 to 5,
      R_{L}¹ is halogen, (C₁-C₄)-alkyl, (C₁-C₄)-haloalkyl, (C₁-C₄)-alkoxy, (C₁-C₄)-haloalkoxy, nitro, (C₁-C₄)-alkylthio, (C₁-C₄)-alkylsulfonyl, (C₁-C₄)-alkoxycarbonyl, optionally substituted phenyl, optionally substituted phenoxy,
      R_{L}² is hydrogen or (C₁-C₄)-alkyl,
      R_{L}³ is hydrogen, (C₁-C₈)-alkyl, (C₂-C₄)-alkenyl, (C₂-C₄)-alkynyl or aryl, where each of the carbon-containing radicals mentioned above is unsubstituted or substituted by one or more, preferably by up to three, identical or different radicals from the group consisting of halogen and alkoxy; or salts thereof,
      M) active compounds from the class of the 3-(5-tetrazolylcarbonyl)-2-quinolones, for example
1,2-dihydro-4-hydroxy-1-ethyl-3-(5-tetrazolylcarbonyl)-2-quinolone (CAS Reg. No.: 219479-18-2), 1,2-dihydro-4-hydroxy-1-methyl-3-(5-tetrazolylcarbonyl)-2-quinolone (CAS Reg. No.: 95855-00-8), as described in WO-A-1999000020,
   N) compounds of the formula (S-XI) or (S-XII),
   as described in WO-A-2007023719 and WO-A-2007023764

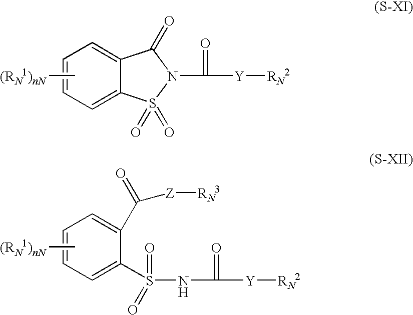

in which
R_{N}¹ is halogen, (C₁-C₄)-alkyl, methoxy, nitro, cyano, CF₃, OCF₃
Y, Z independently of one another are O or S,
n_{N} is an integer from 0 to 4,
R_{N}² is (C₁-C₁₆)-alkyl, (C₂-C₆)-alkenyl, (C₃-C₆)-cycloalkyl, aryl, benzyl, halobenzyl,
R_{N}³ is hydrogen, (C₁-C₆)alkyl,
O) one or more compounds from the group consisting of:
1,8-naphthalic anhydride,
O,O-diethyl S-2-ethylthioethyl phosphorodithioate (disulfoton),
4-chlorophenyl methylcarbamate (mephenate),
O,O-diethyl O-phenyl phosphorothioate (dietholate),
4-carboxy-3,4-dihydro-2H-1-benzopyran-4-acetic acid (CL-304415, CAS Reg. No.: 31541-57-8),
2-propenyl 1-oxa-4-azaspiro[4.5]decane-4-carbodithioate (MG-838, CAS Reg. No.: 133993-74-5),
methyl [(3-oxo-1H-2-benzothiopyran-4(3H)-ylidene)methoxy]acetate (from WO-A-98/13361; CAS Reg. No.: 205121-04-6),
cyanomethoxyimino(phenyl)acetonitrile (cyometrinil),
1,3-dioxolan-2-ylmethoxyimino(phenyl)acetonitrile (oxabetrinil),
4′-chloro-2,2,2-trifluoroacetophenone O-1,3-dioxolan-2-ylmethyloxime (fluxofenim),
4,6-dichloro-2-phenylpyrimidine (fenclorim),
benzyl 2-chloro-4-trifluoromethyl-1,3-thiazole-5-carboxylate (flurazole),
2-dichloromethyl-2-methyl-1,3-dioxolane (MG-191),
   including the stereoisomers, and the salts customary in agriculture.

A mixture with other known active compounds, such as fungicides, insecticides, acaricides, nematicides, bird repellents, plant nutrients and soil structure improvers is likewise possible.

Some of the safeners are already known as herbicides and accordingly, in addition to the herbicidal action against harmful plants, also act by protecting the crop plants. The weight ratios of herbicide (mixture) to safener generally depend on the herbicide application rate and the effectiveness of the safener in question and may vary within wide limits, for example in the range from 200:1 to 1:200, preferably from 100:1 to 1:100, in particular from 20:1 to 1:20. The safeners may be formulated analogously to the compounds of the formula (I) or their mixtures with other herbicides/pesticides and be provided and used as a finished formulation or as a tank mix with the herbicides.

The required application rate of the compound of the formula (I) varies depending, inter alia, on external conditions such as temperature, humidity and the type of herbicide used. It can vary within wide limits, for example between 0.001 and 10 000 g/ha or more of active substance; however, it is preferably between 0.5 and 5000 g/ha, particularly preferably between 0.5 and 1000 g/ha and very particularly preferably between 0.5 and 500 g/ha.

The active compounds according to the invention can be used, for example, in connection with the following plants:

dicotyledonous weeds of genera: Abutilon, Amaranthus, Ambrosia, Anoda, Anthemis, Aphanes, Atriplex, Bellis, Bidens, Capsella, Carduus, Cassia, Centaurea, Chenopodium, Cirsium, Convolvulus, Datura, Desmodium, Emex, Erysimum, Euphorbia, Galeopsis, Galinsoga, Hibiscus, Ipomoea, Kochia, Lamium, Lepidium, Lindernia, Matricaria, Mentha, Mercurialis, Mullugo, Myosotis, Papaver, Pharbitis, Plantago, Polygonum, Portulaca, Ranunculus, Raphanus, Rorippa, Rotala, Rumex, Salsola, Senecio, Sesbania, Sida, Sinapis, Solanum, Sonchus, Sphenoclea, Stellaria, Taraxacum, Thlaspi, Trifolium, Urtica, Veronica, Viola, Xanthium.

Dicotyledonous crops of the genera: Arachis, Cucumis, Cucurbita, Daucus, Glycine, Gossypium, Linum, Lycopersicon, Nicotiana, Pisum, Solanum, Vicia.

Monocotyledonous weeds of the genera: Aegilops, Agropyron, Agrostis, Alopecurus, Apera, Avena, Brachiaria, Bromus, Cenchrus, Commelina, Cynodon, Cyperus, Dactyloctenium, Digitaria, Echinochloa, Eleocharis, Eleusine, Eragrostis, Eriochloa, Festuca, Fimbristylis, Heteranthera, Imperata, Ischaemum, Leptochloa, Lolium, Monochoria, Panicum, Paspalum, Phalaris, Phleum, Poa, Rottboellia, Sagittaria, Scirpus, Setaria, Sorghum.

Monocotyledonous crops of the genera: Allium, Ananas, Asparagus, Avena, Hordeum, Oryza, Panicum, Saccharum, Secale, Sorghum, Triticale, Triticum, Zea.

However, the use of the active compounds according to the invention is in no way restricted to these genera, but also extends in the same manner to other plants.

The salts according to the invention, in particular the compounds of the formula (I), are also suitable, depending on the concentration, for the total control of weeds, for example on industrial terrain and rail tracks, and on paths and areas with or without tree plantings. Similarly, the active compounds according to the invention can be employed for controlling weeds in perennial crops, for example forests, decorative tree plantings, orchards, vineyards, citrus groups, nut orchards, banana plantations, coffee plantations, tea plantations, rubber plantations, oil palm plantations, cocoa plantations, soft fruit plantings and hop fields, on lawns, turf and pastureland, and for the selective control of weeds in annual crops.

The salts according to the invention, in particular the compounds of the formula (I), have strong herbicidal activity and a broad activity spectrum when used on the soil and on above-ground parts of plants. To a certain extent, they are also suitable for the selective control of monocotyledonous and dicotyledonous weeds in monocotyledonous and dicotyledonous crops, both by the pre-emergence and by the post-emergence method, and by sequential application.

The salts according to the invention, in particular the compounds of the formula (I), have a favorable effect on follower crops (rotating behavior), i.e. an extremely low, if any, phytotoxicity (such as, for example, in the form of (a) light-green to yellow leaf veins, (b) yellowing of entire plants, (c) delayed plant growth, (d) abnormal development of younger plant parts or the entire plant) on various follower crops sensitive to the salts according to the invention, in particular to the compounds of the formula (I), such as, for example, sugar beet, sunflower or cruciferous plants, such as oilseed rape, mustard and wild turnips, has been observed.

The preparation and the use of the active compounds according to the invention is illustrated by the examples below.

### A. SYNTHESIS EXAMPLES

### 2-Iodobenzenesulfonamide

At −70° C., 61.5 ml (98.45 mmol) of a 1.6 molar n-butyllithium solution in tetrahydrofuran are added slowly to a solution of 10 g (46.88 mmol) of N-tert-butylbenzenesulfonamide in 150 ml of anhydrous tetrahydrofuran. The solution is then briefly warmed to −30° C. and once more cooled to −70° C., and 13.09 g (51.57 mmol) of iodine, dissolved in 80 ml of tetrahydrofuran, are then added. After this addition, the solution is slowly warmed to room temperature and stirred at this temperature for 3 h. The solution is then washed with 50% strength aqueous sodium thiosulfate solution and with water. The organic phase is dried and evaporated. This gives 4.3 g (27% of theory) of N-tert-butyl-2-iodobenzenesulfonamide.

At room temperature, 4.3 g (12.68 mmol) of N-tert-butyl-2-iodobenzenesulfonamide are stirred in 15 ml of trifluoroacetic acid for 6 h. The solid obtained after evaporation of the solution is washed with water. This gives 3.1 g (86% of theory) of 2-iodobenzenesulfonamide.

### 2-Iodobenzenesulfonyl isocyanate

2.1 g (21.2 mmol) of n-butyl isocyanate and 0.04 g (0.35 mmol) of 1,4-diaza-bicyclo[2.2.2]octane are added to a solution of 5.0 g (17.66 mmol) of 2-iodobenzenesulfonamide in 50 ml of xylene, and the mixture is stirred at 150° C. (reflux) for 1 h. At 120-125° C., a solution of 2.27 g (11.48 mmol) of trichloromethyl chloroformate in 10 ml of xylene is then added dropwise over a period of 1.5 h. The mixture is then stirred at 150° C. for 1 h. Finally, the solvent and all volatile components are removed by distillation under reduced pressure. The residue (5.0 g) is used without further purification for subsequent reactions.

2-Iodobenzenesulfonyl isocyanate can be detected by IR spectroscopy owing to the presence of a strong NCO vibrational band at 2238 cm⁻¹.

1H-NMR data: (400 MHz, CDCl₃, δ, ppm): 8.19 (dd, J=1.6, 7.6, 1H); 8.17 (dd, J=1.6, 7.6, 1H); 7.57 (dt, J=1.6, 8.0, 1H); 7.33 (dt, J=2.0, 8.0, 1H)

### 2-Iodo-N-[(4-methoxy-6-methyl-1,3,5-triazin-2-yl)carbamoyl]benzenesulfonamide

250 mg (0.88 mmol) of 2-iodobenzenesulfonamide and 253 mg (0.97 mmol) of 2-(N-phenyloxycarbonyl)amino-4-methoxy-6-methyl-1,3,5-triazine are initially charged in 3 ml of acetonitrile. 0.26 ml (1.77 mmol) of 1,8-diazabicyclo[5.4.0]undec-7-ene is then added, and the solution is stirred at room temperature for 1 h. Using 2N hydrochloric acid, the solution is then slowly adjusted to pH 1. The precipitated solid is filtered off with suction, washed with diisopropyl ether and dried. This gives 360 mg (90% of theory) of 2-iodo-N-[(4-methoxy-6-methyl-1,3,5-triazin-2-yl)carbamoyl]benzenesulfonamide.

### 2-Iodo-N-[(4-methoxy-6-methyl-1,3,5-triazin-2-yl)carbamoyl]benzenesulfonamide sodium salt (=Compound I-2 of Table 1 Below)

0.47 g (11.69 mmol) of sodium hydroxide is added to a solution of 5.0 g (11.13 mmol) of 2-iodo-N-[(4-methoxy-6-methyl-1,3,5-triazin-2-yl)carbamoyl]-benzenesulfonamide in 500 ml of acetonitrile and 50 ml of water. The solution is stirred at room temperature overnight. After evaporation of the solvent and drying under high vacuum, the sodium salt is obtained in quantitative yield.

1H-NMR data: (300 MHz, d₆-DMSO, δ, ppm): 8.97 (br. s, 1H); 8.03 (dd, J=1.7, 7.9, 1H); 7.94 (dd, J=1.1, 7.8, 1H); 7.44 (dt, J=1.2, 7.4, 1H); 7.10 (dt, J=1.7, 7.4, 1H); 3.83 (s, 3H); 2.29 (s, 3H).

The following compounds are obtained in an analogous manner:

### 2-Iodo-N-[(4-methoxy-6-methyl-1,3,5-triazin-2-yl)carbamoyl]benzenesulfonamide potassium salt (=Compound I-3 of Table 1 Below) by Reaction with Potassium Hydroxide

1H-NMR data: (300 MHz, d₆-DMSO, δ, ppm): 9.06 (br. s, 1H); 8.04 (dd, J=1.6, 7.9, 1H); 7.94 (dd, J=1.0, 7.8, 1H); 7.45 (dt, J=1.2, 7.7, 1H); 7.11 (dt, J=1.5, 7.6, 1H); 3.84 (s, 3H); 2.29 (s, 3H).

### 2-Iodo-N-[(4-methoxy-6-methyl-1,3,5-triazin-2-yl)carbamoyl]benzenesulfonamide lithium salt (=compound I-1 of Table 1 Below) by Reaction with Lithium Hydroxide

1H-NMR data: (300 MHz, d₆-DMSO, δ, ppm): 9.56 (br. s, 1H); 8.05 (dd, J=1.6, 7.9, 1H); 7.95 (dd, J=1.1, 7.8, 1H); 7.46 (ddd, J=1.2, 7.4, 7.8, 1H); 7.11 (dt, J=1.7, 7.7, 1H); 3.86 (s, 3H); 2.30 (s, 3H).

### 2-Iodo-N-[(4-methoxy-6-methyl-1,3,5-triazin-2-yl)carbamoyl]benzenesulfonamide magnesium salt (=compound I-4 of Table 1 Below) by Reaction with Magnesium Hydroxide

1H-NMR data: (300 MHz, d₆-DMSO, δ, ppm): 8.96 (br. s, 1H); 8.05 (dd, J=1.6, 7.9, 1H); 7.94 (dd, J=1.1, 7.8, 1H); 7.44 (dt, J=1.2, 7.7, 1H); 7.10 (dt, J=1.7, 7.5, 1H); 3.84 (s, 3H); 2.30 (s, 3H).

### 2-Iodo-N-[(4-methoxy-6-methyl-1,3,5-triazin-2-yl)carbamoyl]benzenesulfonamide calcium salt (=compound I-5 of Table 1 Below) by Reaction with Calcium Hydroxide

1H-NMR data: (300 MHz, d₆-DMSO, δ, ppm): 8.87 (br. s, 1H); 8.02 (dd, J=1.7, 7.9, 1H); 7.93 (dd, J=1.2, 7.8, 1H); 7.43 (dt, J=1.2, 7.7, 1H); 7.08 (dt, J=1.7, 7.6, 1H); 3.83 (s, 3H); 2.28 (s, 3H).

### 2-Iodo-N-[(4-methoxy-6-methyl-1,3,5-triazin-2-yl)carbamoyl]benzenesulfonamide-(2-hydroxyeth-1-yl)ammonium salt (=Compound I-19 of Table 1 Below) by Reaction with 2-aminoethanol

1H-NMR data: (300 MHz, d₆-DMSO, δ, ppm): 8.03 (dd, J=1.7, 7.9, 1H); 7.93 (dd, J=1.2, 7.8, 1H); 7.71 (br. s, 1H); 7.44 (dt, J=1.2, 7.8, 1H); 7.09 (dt, J=1.7, 7.6, 1H); 5.11 (br. s, 1H); 3.83 (s, 3H); 3.57 (t, J=5.3, 2H); 2.85 (t, J=5.5, 2H); 2.28 (s, 3H).

### 2-Iodo-N-[(4-methoxy-6-methyl-1,3,5-triazin-2-yl)carbamoyl]benzenesulfonamide bis-N,N-(2-hydroxyeth-1-yl)ammonium salt (=Compound I-20 of Table 1 Below) by Reaction with 2,2′-Iminodiethanol

1H-NMR data: (300 MHz, d₆-DMSO, δ, ppm): 8.02 (dd, J=1.7, 7.9, 1H); 7.93 (dd, J=1.2, 7.8, 1H); 7.43 (dt, J=1.2, 7.7, 1H); 7.08 (dt, J=1.7, 7.5, 1H); 5.13 (br. s, 2H); 3.83 (s, 3H); 3.64 (t, J=5.2, 4H); 2.98 (t, J=5.5, 4H); 2.28 (s, 3H).

### 2-Iodo-N-[(4-methoxy-6-methyl-1,3,5-triazin-2-yl)carbamoyl]benzenesulfonamide tris-N,N,N-(2-hydroxyeth-1-yl)ammonium salt (=Compound I-21 of Table 1 Below) by Reaction with 2,2,2′″-nitrilotriethanol

1H-NMR data: (300 MHz, d₆-DMSO, δ, ppm): 9.05 (br. s, 1H); 8.05 (dd, J=1.4, 7.8, 1H); 7.97 (br. d, J=8.1, 1H); 7.47 (br. t, J=7.6, 1H); 7.14 (br. t, J=8.2, 1H); 4.99 (br. s, 3H); 3.86 (s, 3H); 3.65 (br. s, 6H); 3.08 (br. s, 6H); 2.31 (s, 3H).

### 2-Iodo-N-[(4-methoxy-6-methyl-1,3,5-triazin-2-yl)carbamoyl]benzenesulfonamide ammonium salt (=Compound I-6 of Table 1 Below) by Reaction with Ammonia

1H-NMR data: (300 MHz, d₆-DMSO, δ, ppm): 9.33 (br. s, 1H); 8.08 (dd, J=1.6, 7.9, 1H); 8.00 (d, J=7.6, 1H); 7.50 (t, J=7.8, 1H); 7.18 (br. t, J=7.3, 1H); 7.12 (br. s, 3H); 3.87 (s, 3H); 2.34 (s, 3H).

### 2-Iodo-N-[(4-methoxy-6-methyl-1,3,5-triazin-2-yl)carbamoyl]benzenesulfonamide isopropylammonium salt (=Compound I-15 of Table 1 Below) by Reaction with Isopropylamine

1H-NMR data: (300 MHz, d₆-DMSO, δ, ppm): 9.06 (br. s, 1H); 8.04 (dd, J=1.6, 7.9, 1H); 7.95 (dd, J=0.9, 7.7, 1H); 7.63 (br s, 3H); 7.46 (dt, J=1.0, 7.8, 1H); 7.12 (dt, J=1.1, 7.4, 1H); 3.84 (s, 3H); 3.26 (spt, J=6.6, 1H); 2.30 (s, 3H); 1.15 (d, J=6.5, 6H).

### 2-Iodo-N-[(4-methoxy-6-methyl-1,3,5-triazin-2-yl)carbamoyl]benzenesulfonamide tetraethylammonium salt (=Compound I-12 of Table 1 Below) by Reaction with Tetraethylammonium Hydroxide

1H-NMR data: (300 MHz, d₆-DMSO, δ, ppm): 8.78 (br. s, 1H); 8.02 (dd, J=1.6, 7.9, 1H); 7.93 (dd, J=1.1, 7.8, 1H); 7.43 (ddd, J=1.3, 7.4, 7.8, 1H); 7.08 (ddd, J=1.7, 7.4, 7.7, 1H); 3.82 (s, 3H); 3.19 (m, 8H); 2.27 (s, 3H); 1.15 (m, 12H).

### 2-Iodo-N-[(4-methoxy-6-methyl-1,3,5-triazin-2-yl)carbamoyl]benzenesulfonamide tetrapropylammonium salt (=Compound I-14 of Table 1 Below) by Reaction with Tetrapropylammonium Hydroxide

1H-NMR data: (300 MHz, d₆-DMSO, δ, ppm): 8.76 (br. s, 1H); 8.02 (dd, J=1.6, 7.9, 1H); 7.92 (dd, J=1.1, 7.8, 1H); 7.43 (ddd, J=1.3, 7.4, 7.8, 1H); 7.08 (ddd, J=1.7, 7.4, 7.7, 1H); 3.82 (s, 3H); 3.12 (m, 8H); 2.27 (s, 3H); 1.60 (m, 8H); 0.89 (t, J=7.3, 12H).

### 2-Iodo-N-[(4-methoxy-6-methyl-1,3,5-triazin-2-yl)carbamoyl]benzenesulfonamide tetrabutylammonium salt (=Compound I-18 of Table 1 Below) by Reaction with Tetrabutylammonium Hydroxide

1H-NMR data: (300 MHz, d₆-DMSO, δ, ppm): 8.76 (br. s, 1H); 8.02 (dd, J=1.7, 7.9, 1H); 7.92 (dd, J=1.2, 7.8, 1H); 7.42 (dt, J=1.3, 7.7, 1H); 7.07 (dt, J=1.7, 7.6, 1H); 3.82 (s, 3H); 3.16 (m, 8H); 2.26 (s, 3H); 1.57 (m, 8H); 1.31 (m, 8H); 0.93 (t, J=7.5, 12H).

The compounds described in Table 1 below are obtained as described directly above, or analogously to the above examples.

TABLE 1
Compounds of the general formula (I),
where M⁺ denotes the respective salt of the compound

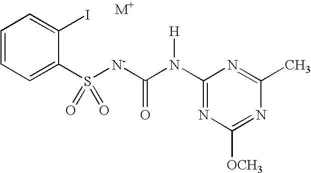

| Compound | M⁺ |
|---|---|
| I-1 | lithium |
| I-2 | sodium |
| I-3 | potassium |
| I-4 | magnesium |
| I-5 | calcium |
| I-6 | ammonium |
| I-7 | methylammonium |
| I-8 | dimethylammonium |
| I-9 | tetramethylammonium |
| I-10 | ethylammonium |
| I-11 | diethylammonium |
| I-12 | tetraethylammonium |
| I-13 | propylammonium |
| I-14 | tetrapropylammonium |
| I-15 | isopropylammonium |
| I-16 | diisopropylammonium |
| I-17 | butylammonium |
| I-18 | tetrabutylammonium |
| I-19 | (2-hydroxyeth-1-yl)ammonium |
| I-20 | bis-N,N-(2-hydroxyeth-1-yl)ammonium |
| I-21 | tris-N,N,N-(2-hydroxyeth-1-yl)ammonium |
| I-22 | 1-phenylethylammonium |
| I-23 | 2-phenylethylammonium |
| I-24 | trimethylsulfonium |
| I-25 | trimethyloxonium |
| I-26 | pyridinium |
| I-27 | 2-methylpyridinium |
| I-28 | 4-methylpyridinium |
| I-29 | 2,4-dimethylpyridinium |
| I-30 | 2,6-dimethylpyridinium |
| I-31 | piperidinium |
| I-32 | imidazolium |
| I-33 | morpholinium |
| I-34 | 1,5-diazabicyclo[4.3.0]non-7-enium |
| I-35 | 1,8-diazabicyclo[5.4.0]undec-7-enium |

### B. FORMULATION EXAMPLES

A dust is obtained by mixing 10 parts by weight of a salt according to the invention, in particular a compound of the formula (I), and 90 parts by weight of talc as inert substance and comminuting the mixture in a hammer mill.
b) A wettable powder which is readily dispersible in water is obtained by mixing 25 parts by weight of a salt according to the invention, in particular a compound of the formula (I), 64 parts by weight of kaolin-containing quartz as inert material, 10 parts by weight of potassium lignosulfonate and 1 part by weight of sodium oleoylmethyltauride as wetting agent and dispersant, and grinding the mixture in a pinned-disk mill.
c) A dispersion concentrate which is readily dispersible in water is obtained by mixing 20 parts by weight of a salt according to the invention, in particular a compound of the formula (I) according to the invention, with 6 parts by weight of alkylphenol polyglycol ether (®Triton X 207), 3 parts by weight of isotridecanol polyglycol ether (8 EO) and 71 parts by weight of paraffinic mineral oil (boiling range for example approx. 255 to above 277° C.), and grinding the mixture in a ball mill to a fineness of below 5 micron.
d) An emulsifiable concentrate is obtained from 15 parts by weight of a compound of the formula (I), 75 parts by weight of cyclohexanone as solvent and 10 parts by weight of oxethylated nonylphenol as emulsifier.
e) Water-dispersible granules are obtained by mixing
   75 parts by weight of a compound according to the invention, in particular a compound of the formula (I),
   10 parts by weight of calcium lignosulfonate,
   5 parts by weight of sodium lauryl sulfate,
   3 parts by weight of polyvinyl alcohol and
   7 parts by weight of kaolin,
   grinding the mixture in a pinned-disk mill and granulating the powder in a fluidized bed by spraying on water as granulation liquid.
f) Water-dispersible granules are also obtained by homogenizing and precomminuting, in a colloid mill,
   25 parts by weight of a compound according to the invention, in particular a compound of the formula (I),
   5 parts by weight of sodium 2,2′-dinaphthylmethane-6,6′-disulfonate,
   2 parts by weight of sodium oleoylmethyltauride,
   1 part by weight of polyvinyl alcohol,
   17 parts by weight of calcium carbonate and
   50 parts by weight of water,
   subsequently grinding the mixture in a bead mill, and atomixing and drying the resulting suspension in a spray tower by means of a single-substance nozzle.

### C. BIOLOGICAL EXAMPLES

### 1. Pre-Emergence Herbicidal Action

Seeds or rhizome pieces of mono- and dicotyledonous weeds were placed in sandy loam in cardboard pots and covered with soil. The compounds according to the invention, formulated in the form of wettable powders or emulsion concentrates, were then applied to the surface of the covering soil in the form of aqueous suspensions or emulsions at an application rate of 100 to 800 l of water/ha (converted), at various dosages.

After the treatment, the pots were placed in a greenhouse and kept under good growth conditions for the weeds. The visual scoring of the damage to the plants or the emergence damage was carried out after the emergence of the test plants after a test period of 3 to 4 weeks, by comparison with untreated controls. As shown by the results, the compounds according to the invention have good herbicidal pre-emergence activity against a broad spectrum of weed grasses and broad-leaved weeds.

For example, the compounds Nos. I-1, I-2, I-3, I-4, I-12, I-18, I-19 from Table 1 have very good herbicidal activity against harmful plants such as Matricaria inodora, Papaver rhoeas, Stellaria media and Viola tricolor when applied by the pre-emergence method at an application rate of 0.08 kg or less of active substance per hectare.

### 2. Post-Emergence Herbicidal Action—Greenhouse

Seeds or rhizome pieces of monocotyledonous and dicotyledonous weeds were placed in sandy loam in plastic pots, covered with soil and cultivated in a greenhouse under good growth conditions. Three weeks after sowing, the test plants were treated at the three-leaf stage. The compounds according to the invention, formulated as wettable powders or as emulsion concentrates, were sprayed onto the green parts of the plants at various dosages using a water application rate of from 100 to 800 l/ha (converted). After the test plants had been left to stand in the greenhouse for about 10 to 28 days under optimum growth conditions, the activity of the preparations was scored visually in comparison to untreated controls. Applied by the post-emergence method, the compositions according to the invention likewise have good herbicidal activity against a broad spectrum of economically important weed grasses and broad-leaved weeds.

For example, the compounds Nos. I-1, I-2, I-3, I-4, I-12, I-18, I-19 from Table 1 have very good herbicidal activity against harmful plants such as Amaranthus retroflexus, Lolium multiflorum, Abuthilon theophrasti, Matricaria inodora, Ipomoea purpurea, Panicum minor, Stellaria media, Solanum nigrum, Veronica persica and Viola tricolor when applied by the post-emergence method at an application rate of 0.08 kg or less of active substance per hectare.

### 3. Post-Emergence Herbicidal Action—Field Trials

### Test Description

In a direct comparison, under field trial conditions (time of application 3-5-leaf stage of the weeds examined), the activities of the weed control of the free acid “2-iodo-N-[(4-methoxy-6-methyl-1,3,5-triazin-2-yl)carbamoyl]benzenesulfonamide” known from DE 27 15 786 were compared to individual salts according to the invention of 2-iodo-N-[(4-methoxy-6-methyl-1,3,5-triazin-2-yl)carbamoyl]benzenesulfonamide (compound I-3 (potassium salt of 2-iodo-N-[(4-methoxy-6-methyl-1,3,5-triazin-2-yl)carbamoyl]benzenesulfonamide) and 1-4 (magnesium salt of 2-iodo-N-[(4-methoxy-6-methyl-1,3,5-triazin-2-yl)carbamoyl]benzenesulfonamide)), evaluation being carried out 25 days after application. With respect to the activity, both a representative of a monocotyledonous weed (Panicum minor) and a representative of a dicotyledonous weed (Solanum nigrum) were scored according to the scheme below:

0%=no control
100%=complete control

Table A-1 below shows the results obtained, which clearly demonstrate the improved weed control by the compounds of the formula (I) according to the invention, both with respect to monocotyledonous weeds and to dicotyledonous weeds.

TABLE A-1

| | | Solanum | Panicum |
|---|---|---|---|
| | Amount of active | nigrum | minor |
| | compound | control | control |
| Compound | g of ai/ha | in % | in % |
| 2-iodo-N-[(4-methoxy-6- | 20 | 70 | 0 |
| methyl-1,3,5-triazin-2- | | | |
| yl)carbamoyl]- | | | |
| benzenesulfonamide | | | |
| I-3 | 20 (acid equivalent) | 84 | 30 |
| I-4 | 20 (acid equivalent) | 85 | 48 |

In a further direct comparison, under outdoor trial conditions (time of application 2-4-leaf stage of the weeds examined), the activities of the weed control of the free acid “2-iodo-N-[(4-methoxy-6-methyl-1,3,5-triazin-2-yl)carbamoyl]benzene-sulfonamide” known from DE 27 15 786 were compared to the inventive Na⁺ salt of 2-iodo-N-[(4-methoxy-6-methyl-1,3,5-triazin-2-yl)carbamoyl]benzenesulfonamide (compounds I-2 according to the invention) at various application rates, evaluation being carried out 28 days after application. With respect to the activity, the weeds Chenopodium album, Fagopyran repens and Pennisetum glauca were scored according to the scheme below:

0%=no control
100%=complete control

Table A-2 below shows the results obtained, which clearly demonstrate the improved weed control by the compound I-2 according to the invention, compared to the free acid.

TABLE A-2

| | Amount of active | Chenopodium | Fagopyran | Pennisetum |
|---|---|---|---|---|
| | compound | album | repens | glauca |
| Compound | g of ai/ha | Control in % | Control in % | Control in % |
| 2-iodo-N-[(4-methoxy-6- | 15 | 90 | 25 | |
| methyl-1,3,5-triazin-2yl)- | | | | |
| carbamoyl]benzene- | | | | |
| sulfonamide | | | | |
| I-2 | 15 | 93 | 30 | |
| | (acid equivalent) | | | |
| 2-iodo-N-[(4-methoxy-6- | 30 | 98 | 40 | 80 |
| methyl-1,3,5-triazin-2yl)- | | | | |
| carbamoyl]benzene- | | | | |
| sulfonamide | | | | |
| I-2 | 30 | 100 | 45 | 85 |
| | (acid equivalent) | | | |

### 4. Compatibility with Crop Plants

In further tests in the greenhouse, seeds of a relatively large number of crop plants and weeds were placed in sandy loam and covered with soil. Some of the pots were immediately treated as described in section 1, the others were placed in a greenhouse until the plants had developed two to three true leaves and were then sprayed with various dosages of the compounds according to the invention as described in section 2. Four to five weeks after the application and residence in the greenhouse, it was found by visual scoring that even high active compound dosages of the compounds according to the invention, applied by the pre- and post-emergence method, did not damage graminaceous crops, such as barley, oats, rye or wheat. Some of the salts according to the invention demonstrated high selectivity, and they are therefore suitable for controlling unwanted vegetation in agricultural crops.

### 5. Replanting Behavior

In further tests, various dosages of the test substances were worked into the soil. Subsequently, various crop plants were sown in plastic pots filled with the treated soil and cultivated in a greenhouse using a day/night rhythm of 22° C./14° C. After four weeks, the plants were scored according to the scheme below.

0%=no damage
100%=complete damage

Tables B1-1 to B1-5 contain the results of tests with sugar beet using different application rates of (a) the compound I-2 according to the invention and (b) metsulfuron-methyl.

TABLE B1-1

| | Amount of | |
|---|---|---|
| | active | Sugar beet |
| | compound | damage |
| Compound | g of ai/ha | in % |
| I-2 | 30 | 92 |
| metsulfuron-methyl | 30 | 94 |

TABLE B1-2

| | Amount of | |
|---|---|---|
| | active | Sugar beet |
| | compound | damage |
| Compound | g of ai/ha | in % |
| I-2 | 7.5 | 85 |
| metsulfuron-methyl | 7.5 | 90 |

TABLE B1-3

| | Amount of | |
|---|---|---|
| | active | Sugar beet |
| | compound | damage |
| Compound | g of ai/ha | in % |
| I-2 | 1.9 | 61 |
| metsulfuron-methyl | 1.9 | 85 |

TABLE B1-4

| | Amount of | |
|---|---|---|
| | active | Sugar beet |
| | compound | damage |
| Compound | g of ai/ha | in % |
| I-2 | 0.5 | 1 |
| metsulfuron-methyl | 0.5 | 18 |

TABLE B1-5

| | Amount of | |
|---|---|---|
| | active | Sugar beet |
| | compound | damage |
| Compound | g of ai/ha | in % |
| I-2 | 0.1 | 0 |
| metsulfuron-methyl | 0.1 | 11 |

Tables B2-1 to B2-5 contain the results of tests with oilseed rape (Brassica napus) using different application rates of (a) the compound I-2 according to the invention and (b) metsulfuron-methyl

TABLE B2-1

| | Amount of | |
|---|---|---|
| | active | Brassica napus |
| | compound | damage |
| Compound | g of ai/ha | in % |
| I-2 | 30 | 94 |
| metsulfuron-methyl | 30 | 96 |

TABLE B2-2

| | Amount of | |
|---|---|---|
| | active | Brassica napus |
| | compound | damage |
| Compound | g of ai/ha | in % |
| I-2 | 7.5 | 83 |
| metsulfuron-methyl | 7.5 | 86 |

TABLE B2-3

| | Amount of | |
|---|---|---|
| | active | Brassica napus |
| | compound | damage |
| Compound | g of ai/ha | in % |
| I-2 | 1.9 | 29 |
| metsulfuron-methyl | 1.9 | 54 |

TABLE B2-4

| | Amount of | |
|---|---|---|
| | active | Brassica napus |
| | compound | damage |
| Compound | g of ai/ha | in % |
| I-2 | 0.5 | 1 |
| metsulfuron-methyl | 0.5 | 11 |

TABLE B2-5

| | Amount of | |
|---|---|---|
| | active | Brassica napus |
| | compound | damage |
| Compound | g of ai/ha | in % |
| I-2 | 0.1 | 0 |
| metsulfuron-methyl | 0.1 | 5 |

Tables B3-1 to B3-5 contain the results of tests with broad bean (Vicia faba) using different application rates of (a) the compound I-2 according to the invention and (b) metsulfuron-methyl.

TABLE B3-1

| | Amount of | |
|---|---|---|
| | active | Vicia faba |
| | compound | damage |
| Compound | g of ai/ha | in % |
| I-2 | 30 | 93 |
| metsulfuron-methyl | 30 | 99 |

TABLE B3-2

| | Amount of | |
|---|---|---|
| | active | Vicia faba |
| | compound | damage |
| Compound | g of ai/ha | in % |
| I-2 | 7.5 | 49 |
| metsulfuron-methyl | 7.5 | 96 |

TABLE B3-3

| | Amount of | |
|---|---|---|
| | active | Vicia faba |
| | compound | damage |
| Compound | g of ai/ha | in % |
| I-2 | 1.9 | 2.5 |
| metsulfuron-methyl | 1.9 | 43 |

TABLE B3-4

| | Amount of | |
|---|---|---|
| | active | Vicia faba |
| | compound | damage |
| Compound | g of ai/ha | in % |
| I-2 | 0.5 | 2.5 |
| metsulfuron-methyl | 0.5 | 8 |

TABLE B3-5

| | Amount of | |
|---|---|---|
| | active | Vicia faba |
| | compound | damage |
| Compound | g of ai/ha | in % |
| I-2 | 0.1 | 2.5 |
| metsulfuron-methyl | 0.1 | 5 |

1. An agrochemically active salt of 2-iodo-N-[(4-methoxy-6-methyl-1,3,5-triazin-2-yl)carbamoyl]benzenesulfonamide.

2. The agrochemically active salt as claimed in claim 1 having the formula (I) 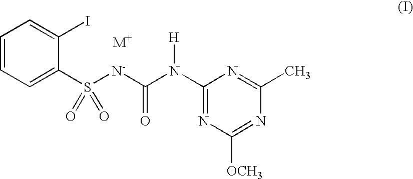
where the cation (M⁺)
(a) is an alkali metal ion, or
(b) is an alkaline earth metal ion, or
(c) is a transition metal ion, or
(d) is an ammonium ion in which optionally one, two, three or all four hydrogen atoms are substituted by identical or different radicals from the group consisting of (C₁-C₄)-alkyl, hydroxy-(C₁-C₄)-alkyl, (C₃-C₆)-cycloalkyl, (C₁-C₄)-alkoxy-(C₁-C₄)-alkyl, hydroxy-(C₁-C₄)-alkoxy-(C₁-C₄)-alkyl, (C₁-C₆)-mercaptoalkyl, phenyl and benzyl, where the radicals mentioned above are optionally substituted by one or more identical or different radicals from the group consisting of halogen, nitro, cyano, azido, (C₁-C₆)-alkyl, (C₁-C₆)-haloalkyl, (C₃-C₆)-cycloalkyl, (C₁-C₆)-alkoxy, (C₁-C₆)-haloalkoxy and phenyl, and where in each case two substituents at the nitrogen atom together optionally form an unsubstituted or substituted ring, or
(e) is a phosphonium ion, or
(f) is a sulfonium ion, or
(g) is an oxonium ion, or
(h) is a saturated or unsaturated/aromatic nitrogenous heterocyclic ionic compound which has 1-10 carbon atoms in the ring system and is optionally mono- or polycondensed and/or mono- or polysubstituted by (C₁-C₄)-alkyl.

3. The compound as claimed in claim 2, wherein the cation (M⁺)
(a) is an alkali metal ion, or
(b) is an alkaline earth metal ion, or
(c) is a transition metal ion, or
(d) is an ammonium ion in which optionally one, two, three or all four hydrogen atoms are substituted by identical or different radicals from the group consisting of (C₁-C₄)-alkyl, hydroxy-(C₁-C₄)-alkyl, (C₃-C₄)-cycloalkyl, (C₁-C₂)-alkoxy-(C₁-C₂)-alkyl, hydroxy-(C₁-C₂)-alkoxy-(C₁-C₂)-alkyl, (C₁-C₂)-mercaptoalkyl, phenyl and benzyl, where the radicals mentioned above are optionally substituted by one or more identical or different radicals from the group consisting of halogen, nitro, cyano, azido, (C₁-C₂)-alkyl, (C₁-C₂)-haloalkyl, (C₃-C₄)-cycloalkyl, (C₁-C₂)-alkoxy, (C₁-C₂)-haloalkoxy and phenyl, and where in each case two substituents at the nitrogen atom together optionally form an unsubstituted or substituted ring, or
(e) is a quaternary phosphonium ion, where the (C₁-C₄)-alkyl radicals and the phenyl radicals are optionally mono- or polysubstituted by identical or different radicals from the group consisting of halogen, (C₁-C₂)-alkyl, (C₁-C₂)-haloalkyl, (C₃-C₄)-cycloalkyl, (C₁-C₂)-alkoxy and (C₁-C₂)-haloalkoxy, or
(f) is a tertiary sulfonium ion, where the (C₁-C₄)-alkyl radicals and the phenyl radicals are optionally mono- or polysubstituted by identical or different radicals from the group consisting of halogen, (C₁-C₂)-alkyl, (C₁-C₂)-haloalkyl, (C₃-C₄)-cycloalkyl, (C₁-C₂)-alkoxy and (C₁-C₂)-haloalkoxy, or
(g) is a tertiary oxonium ion, where the (C₁-C₄)-alkyl radicals are optionally mono- or polysubstituted by identical or different radicals from the group consisting of halogen, (C₁-C₂)-alkyl, (C₁-C₂)-haloalkyl, (C₃-C₄)-cycloalkyl, (C₁-C₂)-alkoxy and (C₁-C₂)-haloalkoxy, or
(h) is a cation from the group of the following heterocyclic compounds, comprising pyridine, quinoline, 2-methylpyridine, 3-methylpyridine, 4-methylpyridine, 2,4-dimethylpyridine, 2,5-dimethylpyridine, 2,6-dimethylpyridine, 5-ethyl-2-methylpyridine, piperidine, pyrrolidine, morpholine, thiomorpholine, pyrrole, imidazole, 1,5-diazabicyclo[4.3.0]non-5-ene (DBN), 1 and or 8-diazabicyclo[5.4.0]undec-7-ene (DBU).

4. The compound as claimed in claim 2, wherein the cation (M⁺) is a sodium ion, a potassium ion, a lithium ion, a magnesium ion, a calcium ion, an NH₄⁺ ion, a (2-hydroxyeth-1-yl)ammonium ion, a bis-N,N-(2-hydroxyeth-1-yl)ammonium ion, a tris-N,N,N-(2-hydroxyeth-1-yl)ammonium ion, a methylammonium ion, a dimethylammonium ion, a trimethylammonium ion, a tetramethylammonium ion, an ethylammonium ion, a diethylammonium ion, a triethylammonium ion, a tetraethylammonium ion, an isopropylammonium ion, a diisopropylammonium ion, a tetrapropylammonium ion, a tetrabutylammonium ion, a 2-(2-hydroxyeth-1-oxy)eth-1-ylammonium ion, a di(2-hydroxyeth-1-yl)ammonium ion, a trimethylbenzylammonium ion, a tri-((C₁-C₄)-alkyl)sulfonium ion or a tri-((C₁-C₄)-alkyl)oxonium ion, a benzylammonium ion, a 1-phenylethylammonium ion, a 2-phenylethylammonium ion, a diisopropylethylammonium ion, a pyridinium ion, a piperidinium ion, an imidazolium ion, a morpholinium ion, and or a 1,8-diazabicyclo[5.4.0]undec-7-enium ion.

5. The compound as claimed in claim 2, wherein the cation (M⁺) is a sodium ion, a potassium ion, a magnesium ion, a calcium ion and or an NH₄⁺ ion.

6. The compound as claimed in claim 2, wherein the cation (M⁺) is a sodium ion and or a potassium ion.

7. A process for preparing a compound as claimed in claim 2 comprising reacting starting materials to form said compound.

8. A method for controlling unwanted vegetation, which comprises allowing at least one compound as claimed in claim 1 to act upon an unwanted plant and/or their habitat thereof.

9. A compound as claimed in claim 1 for controlling unwanted plants.

10. A herbicidal composition, which comprises a compound as claimed in claim 1 and a customary extender and/or surfactant.

11. A 2-iodobenzenesulfonyl isocyanate of the formula (VI)

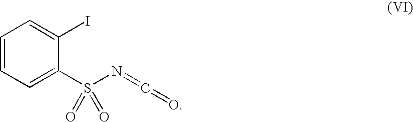

12. A process for preparing 2-iodobenzenesulfonyl isocyanate, which comprises:
reacting iodobenzenesulfonamide of the formula (II) 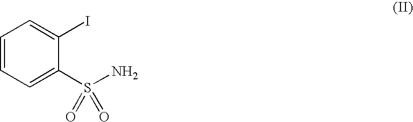
with phosgene, diphosgene or thiophosgene at a temperature 80° C. to 150° C.

13. A herbicidal composition, which comprises a compound as claimed in claim 1 and at least one further agrochemically active compound.
